SEQUENCE LISTING (4 sequences)

SEQ ID NO: 1 - DNA, 4772 bp, Homo sapiens
gctcagttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa

SEQ ID NO: 2 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 3 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 4 - protein, 403 aa, Homo sapiens [sequence omitted]

(19) United States
(12) Patent Application Publication
(10) Pub. No.: US 2014/0179796 A1
(43) Pub. Date: Jun. 26, 2014

(54) MARKERS FOR DCIS RECURRENCE: COORDINATE PTEN/RB LOSS

(71) Applicant: THOMAS JEFFERSON UNIVERSITY, Philadelphia, PA (US)

(72) Inventors: Agnieszka WITKIEWICZ, Philadelphia, PA (US); Erik KNUDSEN, Philadelphia, PA (US); Gordon SCHWARTZ, Philadelphia, PA (US)

(73) Assignee: THOMAS JEFFERSON UNIVERSITY, Philadelphia, PA (US)

(21) Appl. No.: 14/132,137

(22) Filed: Dec. 18, 2013

Related U.S. Application Data
(60) Provisional application No. 61/739,936, filed on Dec. 20, 2012.

(51) Int. Cl. C12Q 1/68; G01N 33/574

(52) U.S. Cl. CPC C12Q 1/6886; G01N 33/574
USPC 514/648; 600/1; 128/898

(57) ABSTRACT

The technology described herein relates to compositions, kits, assays, systems and methods relating to breast cancer and the treatment thereof.

### CROSS-REFERENCE TO RELATED APPLICATIONS

This application claims benefit under 35 U.S.C. §119(e) of U.S. Provisional Application No. 61/739,936 filed Dec. 20, 2012, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Dec. 17, 2013, is named 003252-073861-US_SL.txt and is 25,388 bytes in size

### TECHNICAL FIELD

The technology described herein relates to methods for determining the risk of recurrence in a subject having ductal carcinoma in situ (DCIS).

### BACKGROUND

Ductal carcinoma in situ (DCIS) is a nonobligatory precursor to invasive breast cancer (IBC). With increased use of screening mammography to detect occult breast cancer, the incidence of DCIS markedly increased, and it is estimated that 1 million women will be living with this condition by 2020. Left untreated up to 53% DCIS will progress to invasive breast cancer. Unfortunately, DCIS classifications used in clinical practice do not adequately predict risk of DCIS recurrence and progression. Recently, a new pathologic grading system was proposed to improve prediction of local recurrence (Pinder S E et al. Br J Cancer. 2010; 103(1):94-100) However, examination of cases having what this new system categorized as low-grade DCIS showed that 39.3% of these patients developed invasive breast cancer in the same quadrant as the initial biopsy with most events occurring within 10-15 years, but with some as late as 23-42 years (Sanders M E, et al. Cancer. 2005; 103(12):2481-2484). The results of this study suggest that a subset of patients with low grade DCIS will develop life-threatening invasive carcinoma. Identification of these patients can help to prevent both under and over treatment. Numerous biomarkers have been investigated for risk stratification of patients with DCIS. While markers such as elevated Ki-67 levels, p53 mutations, HER2 amplification, and elevated p16ink4a levels have been studied in the progression of DCIS recurrence and/or progression, none has yet proved clinically useful (Hogue A, et al. Cancer Epidemiol Biomarkers Prev. 2002; 11(6):587-590; Gauthier M L, et al. Cancer Cell. 2007; 12(5):479-491; Cornfield D B, et al. Cancer. 2004; 100(11):2317-2327; Witkiewicz A K, et al. Am J Pathol. 2011179(3):1171-1178: Kerlikowske K, et al. J Natl Cancer Inst. 2011; 102(9):627-637).

### SUMMARY

Embodiments of the technology described herein are based on the discovery that a coordinate decrease of both RB and PTEN expression indicates that a subject with DCIS is significantly more likely to experience a recurrence of DCIS and/or develop invasive breast cancer. In some embodiments, the subject is significantly more likely to experience a recurrence of DCIS and/or develop invasive breast cancer after being treated by lumpectomy, indicating that such subjects can benefit from more aggressive treatment or a combination of treatment options (e.g. mastectomy, radiation therapy, chemotherapy, and/or increased surveillance following administration of a treatment). The expression level of PTEN is not, in and of itself, a statistically significant predictor of clinical outcomes. As demonstrated herein, determination of the expression level of both RB and PTEN in combination provides more than a 30% increase in predictive power as compared to the use of RB alone.

In one aspect, described herein is an assay comprising: subjecting a test sample from a subject to at least one analysis to determine the level of expression of both (a) retinoblastoma 1 (RB) and (b) phosphatase and tensin homolog (PTEN); wherein a decrease in expression of both RB and PTEN relative to a reference level indicates the subject has a higher risk of experiencing an ipsilateral breast event. In some embodiments, a decrease in expression of both RB and PTEN relative to a reference level can indicate the subject has a higher risk of experiencing an ipsilateral breast event which will progress to invasive breast cancer. In some embodiments, the subject can have ductal carcinoma in situ (DCIS). In some embodiments, the expression level of both (a) retinoblastoma 1 (RB) and (b) phosphatase and tensin homolog (PTEN) can be the level of RNA transcript expression product of each gene. In some embodiments, the RNA transcript expression product levels can be assayed using reverse transcription polymerase chain reaction (RT-PCR). In some embodiments, the expression level of both (a) retinoblastoma 1 (RB) and (b) phosphatase and tensin homolog (PTEN) can be the level of polypeptide expression product of each gene. In some embodiments, the polypeptide expression product levels can be assayed using immunohistological staining.

In one aspect, described herein is an assay comprising: (a) obtaining a test sample comprising a ductal carcinoma in situ (DCIS) cell from the breast tissue of a subject by lumpectomy or biopsy; (b) contacting at least one portion of the sample with a primary anti-retinoblastoma 1 (RB) antibody and at least one portion of the sample with a primary anti-phosphatase and tensin homolog (PTEN) antibody; (c) washing the sample to remove excess unbound primary antibody; and (d) detecting the presence or intensity of a detectable signal;

wherein a decrease in the expression level of both RB and PTEN, indicated by the level of the detectable signal, relative to a reference level indicates the subject has a higher risk of experiencing an ipsilateral breast event. In some embodiments, a decrease in expression of both RB and PTEN relative to a reference level can indicate the subject has a higher risk of experiencing an ipsilateral breast event which will progress to invasive breast cancer. In some embodiments, the primary antibody can be detectably labeled or capable of generating a detectable signal. In some embodiments, between steps (c) and (d), the assay can further comprise: contacting the sample with a secondary antibody having a detectable label or capable of generating a detectable signal; and washing the sample to remove excess unbound secondary antibody. In some embodiments, one portion of the sample can be contacted with both a primary anti-retinoblastoma 1 (RB) antibody and a primary anti-phosphatase and tensin homolog (PTEN) antibody. In some embodiments, separate portions of the sample can be contacted with a primary anti-retinoblastoma 1 (RB) antibody and a primary anti-phosphatase and tensin homolog (PTEN) antibody.

In some embodiments, the subject can be at higher risk for experiencing an ipsilateral breast event if the expression levels of both (a) retinoblastoma 1 (RB) and (b) phosphatase and tensin homolog (PTEN) are decreased by at least 50% in the test sample relative to the reference level. In some embodiments, the risk of recurrence can be expressed in terms of a hazard ratio, the likelihood of experiencing an ipsilateral breast event, and/or the likelihood of experiencing an ipsilateral breast event which will progress to invasive breast cancer.

In one aspect, described herein is an assay to determine if a subject with ductal carcinoma in situ (DCIS) is in need of treatment with therapy other than a lumpectomy, the assay comprising: subjecting a test sample of a subject to at least one analysis to determine the level of expression of both (a) retinoblastoma 1 (RB) and (b) phosphatase and tensin homolog (PTEN); wherein a decrease in expression of both RB and PTEN relative to a reference level indicates the subject is in need of treatment with a therapy other than a lumpectomy. In some embodiment, the treatment other than a lumpectomy can be selected from the group consisting of: radiation; chemotherapy; tamoxifen; mastectomy; and radical mastectomy. In some embodiments, the individual can be in need of treatment with a therapy other than a lumpectomy if the expression levels of both (a) retinoblastoma 1 (RB) and (b) phosphatase and tensin homolog (PTEN) are decreased by at least 50% in the test sample relative to the reference level.

In some embodiments, the expression level of both (a) retinoblastoma 1 (RB) and (b) phosphatase and tensin homolog (PTEN) can be normalized relative to the expression level of one or more reference genes. In some embodiments, the test sample can comprise a ductal carcinoma in situ (DCIS) cell. In some embodiments, the test sample can be obtained by performing a lumpectomy or biopsy on the subject. In some embodiments, the reference level of expression of (a) retinoblastoma 1 (RB) and (b) phosphatase and tensin homolog (PTEN) can be the level of expression in non-cancerous tissue of the subject surrounding a ductal carcinoma in situ (DCIS) cell. In some embodiments, the reference level of expression of (a) retinoblastoma 1 (RB) and (b) phosphatase and tensin homolog (PTEN) can be the level of expression in the breast tissue of a healthy subject. In some embodiments, the expression level of no more than 20 other genes is determined. In some embodiments, the expression level of no more than 10 other genes is determined. In some embodiments, the subject can be a human. In some embodiments, the assay can further comprise creating a report based on the expression level of both (a) retinoblastoma 1 (RB) and (b) phosphatase and tensin homolog (PTEN).

In one aspect, described herein is a method of administering a treatment for ductal carcinoma in situ (DCIS) to a subject, the method comprising: subjecting a test sample of a subject to at least one analysis to determine the level of expression of both (a) retinoblastoma 1 (RB) and (b) phosphatase and tensin homolog (PTEN); and administering a treatment for DCIS to the subject if the expression level of both RB and PTEN is decreased relative to a reference level; wherein the treatment is not a lumpectomy. In some embodiments, the aggressive treatment for ductal carcinoma in situ (DCIS) can be selected from the group consisting of: radiation; chemotherapy; tamoxifen; mastectomy; and radical mastectomy.

In one aspect, described herein is a method of identifying a subject with ductal carcinoma in situ (DCIS) who is in need of multiple treatments, the method comprising: subjecting a test sample of a subject to at least one analysis to determine the level of expression of both (a) retinoblastoma 1 (RB) and (b) phosphatase and tensin homolog (PTEN); and wherein the subject is identified as needing multiple treatments for DCIS if the expression level of both RB and PTEN is decreased relative to a reference level. In some embodiments, the multiple treatments for ductal carcinoma in situ (DCIS) comprise: (a) lumpectomy; and (b) at least one further treatment selected from the group consisting of: radiation; chemotherapy; tamoxifen; mastectomy; and radical mastectomy.

In one aspect, described herein is a method of classifying a ductal carcinoma in situ (DCIS) carcinoma, the method comprising: subjecting a test sample of a subject to at least one analysis to determine the level of expression of both (a) retinoblastoma 1 (RB) and (b) phosphatase and tensin homolog (PTEN); and (a) classifying the DCIS as a high risk carcinoma if the expression level of both RB and PTEN is decreased relative to a reference level; (b) classifying the DCIS as a low risk carcinoma if the expression level of both RB and PTEN is not decreased relative to a reference level.

In some embodiments, the expression level of both (a) retinoblastoma 1 (RB) and (b) phosphatase and tensin homolog (PTEN) can be the level of RNA transcript expression product of each gene. In some embodiments, the RNA transcript expression product levels can be assayed using reverse transcription polymerase chain reaction (RT-PCR). In some embodiments, the expression level of both (a) retinoblastoma 1 (RB) and (b) phosphatase and tensin homolog (PTEN) can be the level of polypeptide expression product of each gene. In some embodiments, the polypeptide expression product levels can be assayed using immunohistological staining. In some embodiments, the expression level of both (a) retinoblastoma 1 (RB) and (b) phosphatase and tensin homolog (PTEN) can be normalized relative to the expression level of one or more reference genes. In some embodiments, the test sample can comprise a ductal carcinoma in situ (DCIS) cell. In some embodiments, the test sample can be obtained by performing a lumpectomy or biopsy on the subject. In some embodiments, the reference level of expression of (a) retinoblastoma 1 (RB) and (b) phosphatase and tensin homolog (PTEN) can be the level of expression in non-cancerous tissue of the subject surrounding a DCIS cell. In some embodiments, the reference level of expression of (a) retinoblastoma 1 (RB) and (b) phosphatase and tensin homolog (PTEN) can be the level of expression in the breast tissue of a healthy subject. In some embodiments, the expression level of no more than 20 other genes is determined. In some embodiments, the expression level of no more than 10 other genes is determined. In some embodiments, the subject can be a human.

In one aspect, described herein is a method of determining whether a subject is at risk of experiencing a ipsilateral breast event, the method comprising: determining the level of expression of both (a) retinoblastoma 1 (RB) and (b) phosphatase and tensin homolog (PTEN) in a test sample obtained from a ductal carcinoma in situ (DCIS) cell from a subject; wherein a decrease in expression of both RB and PTEN relative to a reference level indicates the subject has a higher risk experiencing an ipsilateral breast event. In some embodiments, a decrease in expression of both RB and PTEN relative to a reference level can indicate the subject has a higher risk of experiencing an ipsilateral breast event which will progress to invasive breast cancer. In some embodiments, the expression level of both (a) retinoblastoma 1 (RB) and (b) phosphatase and tensin homolog (PTEN) can be the level of RNA transcript expression product of each gene. In some embodiments, the RNA transcript expression product levels can be assayed using reverse transcription polymerase chain reaction (RT-PCR). In some embodiments, the expression level of both (a) retinoblastoma 1 (RB) and (b) phosphatase and tensin homolog (PTEN) can be the level of polypeptide expression product of each gene. In some embodiments, the polypeptide expression product levels can be assayed using immunohistological staining. In some embodiments, the expression level of both (a) retinoblastoma 1 (RB) and (b) phosphatase and tensin homolog (PTEN) can be normalized relative to the expression level of one or more reference genes. In some embodiments, the subject can be at higher risk for experiencing an ipsilateral breast event if the expression levels of both (a) retinoblastoma 1 (RB) and (b) phosphatase and tensin homolog (PTEN) are decreased by at least 50% in the test sample relative to the reference level. In some embodiments, the risk of recurrence can be expressed in terms of a hazard ratio, the likelihood of experiencing an ipsilateral breast event, and/or the likelihood of experiencing an ipsilateral breast event which will progress to invasive breast cancer. In some embodiments, a subject with a higher risk of experiencing an ipsilateral breast event is indicated to receive a treatment selected from the group consisting of: radiation; chemotherapy; tamoxifen; mastectomy; and radical mastectomy. In some embodiments, the reference level of expression of (a) retinoblastoma 1 (RB) and (b) phosphatase and tensin homolog (PTEN) can be the level of expression in non-cancerous tissue of the subject surrounding a ductal carcinoma in situ (DCIS) cell. In some embodiments, the reference level of expression of (a) retinoblastoma 1 (RB) and (b) phosphatase and tensin homolog (PTEN) can be the level of expression in the breast tissue of a healthy subject. In some embodiments, the expression level of no more than 20 other genes is determined. In some embodiments, the expression level of no more than 10 other genes is determined. In some embodiments, the subject can be a human.

In one aspect, described herein is a computer system for determining the risk of a subject experiencing an ipsilateral breast event, the system comprising: a determination module configured to measure the expression level of (a) retinoblastoma 1 (RB) and (b) phosphatase and tensin homolog (PTEN) in a test sample obtained from a subject; a storage module configured to store output data from the determination module; a comparison module adapted to compare the data stored on the storage module with a reference level, and to provide a retrieved content, and a display module for displaying whether RB and PTEN expression products have a statistically significant decrease in expression level in the test sample obtained from a subject as compared to the reference expression level and/or displaying the relative expression levels of the marker gene products. In some embodiments, the measuring module can measure the intensity of a detectable signal from an immunoassay indicating the presence or level of (a) retinoblastoma 1 (RB) and (b) phosphatase and tensin homolog (PTEN) polypeptides in the test sample. In some embodiments, the measuring module can measure the intensity of a detectable signal from a RT-PCR assay indicating the presence or level of (a) retinoblastoma 1 (RB) and (b) phosphatase and tensin homolog (PTEN) RNA transcripts in the test sample. In some embodiments, the reference expression level can be the level of (a) retinoblastoma 1 (RB) and (b) phosphatase and tensin homolog (PTEN) in the breast tissue of a population of healthy subjects. In some embodiments, the reference expression level can be the level of (a) retinoblastoma 1 (RB) and (b) phosphatase and tensin homolog (PTEN) in the healthy breast tissue of the subject with ductal carcinoma in situ (DCIS). In some embodiments, if the computing module determines that the expression level of (a) retinoblastoma 1 (RB) and (b) phosphatase and tensin homolog (PTEN) in the test sample obtained from a subject is lower by a statistically significant amount than the reference expression level, the display module can display a signal indicating that the expression levels in the sample obtained from a subject are lower than those of the reference expression level. In some embodiments, the signal can indicate that the subject has an increased likelihood of experiencing an ipsilateral breast event. In some embodiments, the signal can indicate that the subject has an increased likelihood of experiencing an ipsilateral breast event which will progress to invasive breast cancer. In some embodiments, the signal can indicate the subject is in need of aggressive treatment or multiple forms of treatment. In some embodiments, the signal can indicate the degree to which the expression level of (a) retinoblastoma 1 (RB) and (b) phosphatase and tensin homolog (PTEN) in the sample obtained from a subject vary from the reference expression level.

In one aspect, described herein is a device for measuring the presence or level of (a) retinoblastoma 1 (RB) and (b) phosphatase and tensin homolog (PTEN) expression in a test sample from a subject comprising: (a) at least a RB-specific antibody or antigen-binding portion thereof and a PTEN-specific antibody or antigen-binding portion thereof; and (b) at least one solid support, wherein the antibodies or antigen-binding portions thereof of step a are deposited on the support. In some embodiments, the device can perform an assay in which an antibody-protein or antibody-peptide complex is formed. In some embodiments, the solid support can be in the format of a dipstick, a microfluidic chip, a multi-well plate or a cartridge. In some embodiments, the device can further comprise a reference.

In one aspect, described herein is a kit comprising: a device as described herein; and at least a detection antibody. In some embodiments, the detection antibody can be specific for (a) retinoblastoma 1 (RB) or (b) phosphatase and tensin homolog (PTEN). In some embodiments, the detection antibody can be detectably labeled. In some embodiments, the kit can further comprise at least an agent for producing a detectable signal from the detection antibody.

The details of various embodiments of the technology described herein are set forth in the description below. Other features, objects, and advantages of the invention will be apparent from the description and the drawings, and from the claims.

### DESCRIPTION OF THE DRAWINGS

FIGS. 1A and 1B depict graphs of hazard ratios demonstrating that standard clinicopathological features have little prognostic significance for DCIS: The indicated features were evaluated for association with overall recurrence (1A) or invasive progression (1B). Forest plots show the hazard ratio and the associated 95% confidence intervals. ER=estrogen receptor, PR=progesterone receptor.

FIGS. 2A-2B demonstrate that RB-status is associated with DCIS recurrence and invasive progression. FIG. 2A depicts photomicrographs of RB staining which was extensively optimized. Representative images of stained DCIS lesions are shown. FIG. 2B depicts graphs of Kaplan-Meier analyses performed to determine the association between RB-status and overall recurrence (upper graph) or invasive progression (lower graph).

FIGS. 3A and 3B demonstrate that PTEN-status is not a prognostic marker in DCIS. FIG. 3A depicts photomicrographs of PTEN staining which was extensively optimized. Representative images of stained DCIS lesions are shown. FIG. 3B depicts a graph of Kaplan-Meier analyses performed to determine the association between RB-status and overall recurrence.

FIGS. 4A-4F demonstrate that combined RB and PTEN deficiency is associated with poor outcome in DCIS. FIG. 4A depicts a graph of Kaplan-Meier analyses for all recurrence of all sub-groups based on RB and PTEN staining FIG. 4B depicts a graph of Kaplan-Meier analyses for invasive recurrence of all sub-groups based on RB and PTEN staining FIG. 4C depicts a graph of Kaplan-Meier analyses for all recurrences of RB and PTEN deficient cases vs. all other cases. FIG. 4D depicts a graph of Kaplan-Meier analyses for invasive recurrence of RB and PTEN deficient cases vs. all other cases. FIG. 4E depicts a Forest plot of RB and/or PTEN status and hazard ratios. 95% confidence interval for all recurrence is shown. FIG. 4F depicts a Forest plot of RB and/or PTEN status and hazard ratio. 95% confidence interval for invasive recurrence is shown.

FIG. 5 is a diagram of an embodiment of a system for performing a method for determining the risk of a subject with DCIS, e.g. experiencing an ipsilateral breast event.

FIG. 6 is a diagram of an embodiment of a comparison module as described herein.

FIG. 7 is a diagram of an embodiment of an operating system and applications for a computing system as described herein.

FIGS. 8A-8F demonstrate the functional impact of RB and PTEN deficiency on proliferation and invasion: In FIG. 8A MCF10A models deficient for PTEN and RB were characterized by immunoblotting with the indicated antibodies. FIG. 8B depicts a graph of cell cycle progression of MCF10A variants determined by BrdU incorporation, detection by flow cytometry. Data shown are from triplicate analyses. The mean and standard deviation are shown. FIG. 8C depicts graphs of the ability of PTEN-deficient MCF10A cultures to invade through matrigel in a Boyden chamber assay. Data shown are from independent experiments. The mean and standard deviation are shown relative to the RB-positive control. FIG. 8D depicts representative images of wound healing assays performed with PTEN-deficient MCF10A cells. FIG. 8E depicts a graph of cell cycle progression under low-serum conditions as determined by flow cytometry. Data are from triplicate analyses. The mean and standard deviation are shown. FIG. 8F depicts representative crystal violet staining of cells grown for 3 days under low serum conditions.

FIGS. 9A-9C demonstrate distinct roles for RB and PTEN in controlling growth in 3D. FIG. 9A depicts representative images of the indicated MCF10A cultures that were grown in 3D. Bright field (upper panel) and Immunofluorescent images (lower panel) are shown, with cells stained for Ki67, E-cadherin, and DAPI. FIG. 9B depicts a graph of the quantitation of acinar size as determined by measuring the acinar diameter. The mean and standard deviation are shown. FIG. 9C depicts a graph of the quantitation of Ki67 index was performed from confocal images. The mean and standard deviation are shown.

### DETAILED DESCRIPTION

One aspect of the technology described herein relates to a method of determining the risk of a subject having or diagnosed as having DCIS subsequently experiencing a breast event or developing invasive cancer. The methods described herein can comprise determining the expression of both RB and PTEN in a test sample of the carcinoma. The use of this combination of markers can increase the predictive power of the assay by more than 30% as compared to the use of RB alone, whereas PTEN alone is not a statistically significant predictor of such outcomes. Accordingly, described herein are methods and assays for prognosis of a case of DCIS, methods of treatment, and methods of cancer classification as well as systems, devices, and kits for use in these methods and assays.

For convenience, the meaning of certain terms and phrases used in the specification, examples, and appended claims, are provided below. If there is an apparent discrepancy between the usage of a term in the art and its definition provided herein, the definition provided within the specification shall prevail.

The terms “decrease,” “reduce,” “reduced”, “reduction”, and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference, or any decrease between 10-99% as compared to the reference.

The terms “increased”, “increase” or “enhance” or “activate” are all used herein to generally mean an increase by a statically significant amount; for the avoidance of doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “treat” “treatment” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of cancer progression, delay or slowing of metastasis or invasiveness, and amelioration or palliation of symptoms of DCIS or breast cancer. Treatment also includes a decrease in mortality or an increase in the lifespan of a subject as compared to one not receiving the treatment.

As used herein, the term “administering,” refers to the placement an agent as disclosed herein into a subject by a method or route which results in at least partial localization of the agents at a desired site.

The term “gene” means the nucleic acid sequence (DNA) which is transcribed to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene (e.g. mRNA), and polypeptides obtained by translation of mRNA transcribed from a gene.

As used herein, the termed “proteins” and “polypeptides” are used interchangeably to designate a series of amino acid residues connected to the other by peptide bonds between the alpha-amino and carboxy groups of adjacent residues. The terms “protein”, and “polypeptide”, refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “carcinoma” refers to a cancer arising from epithelial cells. As used herein, the term “invasive” refers to the ability to infiltrate and destroy surrounding tissue. DCIS is not an invasive form of breast cancer.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most, if not all, of these agents are directly toxic to cancer cells and do not require immune stimulation. In one embodiment, a chemotherapeutic agent is an agent of use in treating neoplasms, e.g. such as solid tumors. In one embodiment, a chemotherapeutic agent is a radioactive molecule (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu). One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

As used herein, a “subject” means a human or animal Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, breast cancer. In addition, the methods described herein can be used to treat domesticated animals and/or pets. In some embodiments, the subject is a female subject.

A subject can be one who has been previously diagnosed with or identified as suffering from or having a condition in need of treatment (e.g. DCIS) or one or more complications related to such a condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any tangible storage device used for storing data accessible by a computer, as well as any other means for providing access to data by a computer. A computer readable medium is not a signal. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” can refer to a system having a computer, where the computer comprises a computer-readable medium embodying software to operate the computer.

The term “statistically significant” or “significantly” refers to a standard definition of statistical significance and generally means a two standard deviation (2SD) difference from a reference.

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

Definitions of common terms in cell biology and molecular biology can be found in “The Merck Manual of Diagnosis and Therapy”, 19th Edition, published by Merck Research Laboratories, 2006 (ISBN 0-911910-19-0) and Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); and Current Protocols in Protein Science (CPPS) (John E. Coligan, et. al., ed., John Wiley and Sons, Inc.) which are all incorporated by reference herein in their entireties.

Further definitions are provided in the description of the various embodiments of the technology described below herein.

### DCIS

Most breast cancers are ductal carcinomas, i.e. they originate in the ducts that carry milk to the nipple. Less common are lobular carcinomas, These form in the cells that line the lobules that produce milk. If the cancer cells are confined to the duct or lobule, the cancer is in situ, meaning it hasn't left the site of origin and has not yet become invasive. When a cancer has moved beyond the duct, it is called invasive or infiltrating cancer. The transition from in situ to invasive is a significant change in the structure of the cancerous cells (e.g. the glycans on the surface of the cell, as described by Goetz et al, Glycoconi J. 2009 26:117-31; which is incorporated by reference herein in its entirety) This transition, in addition to reflecting a myriad of structural differences, has important implications for the health of the subject, as invasive cancers pose a much greater risk of mortality and can be harder to treat.

As used herein, “ductal carcinoma in situ” or “DCIS” refers to a noninvasive neoplasm of ductal origin that can, in some cases, progress to invasive cancer. Ductal carcinoma in situ (DCIS) is usually found by mammography, as no tumor mass, or only a small tumor mass, can be present and as a result a woman is unable to find the cancer during breast self-examination. Methods for detecting and diagnosing DCIS are well known in the art (see, e.g. Evans. Breast Cancer Research 2003 5:250-3; which is incorporated by reference herein in its entirety).

In some cases, DCIS can progress to an invasive or infiltrating cancer. As used herein, the phrase “progression to invasive breast cancer” refers to a change in at least one DCIS cell to an invasive or infiltrating phenotype, such that cancerous cells are able to move beyond or grow beyond the duct of origin. Infiltrating ductal carcinoma is the most common type of breast cancer. As the cells invade surrounding areas, scar tissue or other fibrous growth surrounds the tumor cells forming a lump that can be seen on a mammogram or felt during a physician's examination.

When a subject has or is diagnosed as having DCIS, they can undergo treatment to remove and/or destroy the cancerous cells. DCIS lesions were originally treated by mastectomy, but breast conserving surgery with, optionally, radiation therapy and/or hormonal interventions has become a standard treatment. In some cases, following treatment, the subject can display no further signs, symptoms, or markers of DCIS. In some cases, following treatment, the subject can experience an ipsilateral breast event. As used herein, the term “ipsilateral breast event” refers to a second occurrence (i.e. a recurrence) of any type of breast cancer in the same breast as was affected by the original instance of DCIS. In some embodiments, an ipsilateral breast event can be an occurrence of DCIS. In some embodiments, an ipsilateral breast event can be an occurrence of invasive breast cancer. In some embodiments, an ipsilateral breast event can comprise cells which are direct descendents of the original DCIS cells and which were not removed and/or destroyed by treatment. In some embodiments, an ipsilateral breast event can comprise cells which are not direct descendents of the original DCIS cells. i.e., the ipsilateral breast event can be an independent event.

Using current clinical methodologies, it is unclear which subjects will experience a progression to invasive breast cancer and/or experience an ipsilateral breast event or when subjects who do experience these events are likely to do so. Further, it is unclear whether patients who have DCIS uniformly benefit from current treatments. In several studies, radiotherapy reduced in-situ or invasive recurrences by about 50%. Although radiotherapy is associated with substantial reductions in local recurrence, no differences have been reported in overall survival. Furthermore, since only 10-15% of cases recur as invasive disease in the absence of radiation therapy, it is clear that not all DCIS patients require radiation. Similarly, adjuvant tamoxifen treatment can confer decreased risk of contralateral, but not ispsilateral invasive breast cancer, raising the question of effects on recurrence vs. second primary disease. Therefore, identifying DCIS cases that can be cured by surgery alone (e.g. subjects having or diagnosed as having DCIS which are at low risk of experiencing a progression to invasive breast cancer and/or at low risk of experiencing an ipsilateral breast event) can contribute to effectively managing disease and mitigating over-treatment of patients.

### RB/PTEN

In certain embodiments the assays, methods, and systems are directed to determination of the expression level of a gene product of at least RB and PTEN.

The retinoblastoma 1 (RB or RB 1 or pRB) (NCBI Gene ID: 5925 (human)) gene encodes a pocket protein family polypeptide which, when in the active hypophosphorylated state, inhibits cell cycle progression, at least in part by inhibiting E2F transcription factors. RB can also recruit chromatin-remodeling enzymes such as methylases and acteylases and act as part of the DREAM (or LINC) complex. The sequences of the RB gene, mRNA, and polypeptide in a number of species are known; e.g. the human mRNA (SEQ ID NO: 001; NCBI Ref Seq: NM_{—}00321) and polypeptide (SEQ ID NO: 002; NCBI Ref Seq: NP_{—}000312) sequences.

The phosphatase and tensin homolog (PTEN) (NCBI Gene ID: 5728 (human)) gene encodes a phosphatidylinositol-3,4,5-trisphosphate 3-phosphatase which inhibits the progression of the cell cycle and negatively regulates the Akt/PKB pathway. PTEN activity is preferentially directed to dephosphorylation of phosphoinositide, reducing intracellular concentrations of phosphatidylinositol-3,4,5-trisphosphate. PTEN comprises a phosphatase domain comprising the enzymatic active site and a C2 domain to bind the polypeptide to the phospholipid membrane. The sequences of the PTEN gene, mRNA, and polypeptide in a number of species are known; e.g. the human mRNA (SEQ ID NO: 003; NCBI Ref Seq: NM_{—}001314) and polypeptide (SEQ ID NO: 004; NCBI Ref Seq: NP_{—}000305 sequences.

### Assay

As described herein, the inventors have determined that where a subject has DCIS with a decreased level of RB and PTEN, the subject has a higher risk of experiencing an ipsilateral breast event. In some embodiments, the subject has a higher risk of experiencing an ipsilateral breast event which will progress to invasive breast cancer. In some embodiments, the subject has a higher risk than the risk of a reference subject or reference risk level. Accordingly, some embodiments are generally related to assays, methods and systems for assessing the likelihood that a subject having DCIS will experience an ipsilateral breast event and/or an ipsilateral breast event which will progress to invasive breast cancer. In certain embodiments, the assays, methods and systems relate to identifying a subject in need of treatment with multiple treatment methods or with aggressive treatment for DCIS and/or breast cancer. Certain embodiments are related to assays, methods and systems for identifying the risk level of a carcinoma in a test sample obtained from a subject.

In certain embodiments, the assays, methods and systems are directed to determination of the expression level of a gene product (e.g. protein and/or gene transcript such as mRNA) in a biological sample of a subject. In one aspect, the technology described herein relates to an assay comprising subjecting a test sample from a subject to at least one analysis to determine the level of expression of both (a) retinoblastoma 1 (RB) and (b) phosphatase and tensin homolog (PTEN); wherein a decrease in expression of both RB and PTEN relative to a reference level indicates the subject has a higher risk of experiencing an ipsilateral breast event. In some embodiments, a decrease in expression of both RB and PTEN relative to a reference level indicates the subject has a higher risk of experiencing an ipsilateral breast event which will progress to invasive breast cancer.

Subjects who are more likely to experience an ipsilateral breast event and/or an ipsilateral breast event which will progress to invasive breast cancer can benefit from more aggressive treatment. Conversely, subjects who are not likely to experience an ipsilateral breast event and/or an ipsilateral breast even which will progress to invasive breast cancer, are unlikely to benefit, and in fact may needlessly suffer from aggressive treatment. Accordingly, one aspect of the technology described herein relates to an assay to determine if a subject with ductal carcinoma in situ (DCIS) is in need of treatment with therapy other than a lumpectomy, the assay comprising: subjecting a test sample from a subject to at least one analysis to determine the level of expression of both (a) retinoblastoma 1 (RB) and (b) phosphatase and tensin homolog (PTEN); wherein a decrease in expression of both RB and PTEN relative to a reference level indicates the subject is in need of treatment with a therapy other than a lumpectomy. In some embodiments, the treatment other than a lumpectomy is selected from the group consisting of: radiation; chemotherapy; tamoxifen; mastectomy; and radical mastectomy. In some embodiments, the subject can be in need of treatment with a therapy other than a lumpectomy if the expression levels of both (a) retinoblastoma 1 (RB) and (b) phosphatase and tensin homolog (PTEN) are decreased by at least 50% in the test sample relative to the reference level. One aspect of the technology described herein relates to a method of identifying a subject with ductal carcinoma in situ (DCIS) who is in need of multiple treatments, the method comprising: subjecting a test sample of a subject to at least one analysis to determine the level of expression of both (a) retinoblastoma 1 (RB) and (b) phosphatase and tensin homolog (PTEN); and wherein the subject is identified as needing multiple treatments for DCIS if the expression level of both RB and PTEN is decreased relative to a reference level. In some embodiments, multiple treatments for ductal carcinoma in situ (DCIS) can comprise: a) a lumpectomy; and b) at least one further treatment selected from the group consisting of: radiation; chemotherapy; tamoxifen; mastectomy; and radical mastectomy.

In one aspect, provided herein is a method of determining whether a subject is at risk of experiencing a ipsilateral breast event, the method comprising: determining the level of expression of both (a) retinoblastoma 1 (RB) and (b) phosphatase and tensin homolog (PTEN) in a test sample obtained from ductal carcinoma in situ (DCIS) affected tissue from a subject; wherein a decrease in expression of both RB and PTEN relative to a reference level indicates the subject has a higher risk experiencing an ipsilateral breast event and/or experiencing an ipsilateral breast event which will progress to invasive breast cancer. In some embodiments, a subject with a higher risk of experiencing an ipsilateral breast event and/or an ipsilateral breast event which will progress to invasive breast cancer can be indicated to receive a treatment selected from the group consisting of: radiation; chemotherapy; tamoxifen; mastectomy; and radical mastectomy.

Subjects having DCIS cells with reduced expression of RB and PTEN as compared to reference levels are at higher risk of, e.g., experiencing an ipsilateral breast event, and can thus be characterized as having a high risk carcinoma. One aspect of the technology described herein relates to a method of classifying a ductal carcinoma in situ (DCIS) carcinoma, the method comprising: subjecting a test sample of a subject to at least one analysis to determine the level of expression of both (a) retinoblastoma 1 (RB) and (b) phosphatase and tensin homolog (PTEN); and a) classifying the DCIS as a high risk carcinoma if the expression level of both RB and PTEN is decreased relative to a reference level; b) classifying the DCIS as a low risk carcinoma if the expression level of both RB and PTEN is not decreased relative to a reference level.

In one aspect, described herein is a method of administering a treatment for ductal carcinoma in situ (DCIS) to a subject, the method comprising: subjecting a test sample of a subject to at least one analysis to determine the level of expression of both (a) retinoblastoma 1 (RB) and (b) phosphatase and tensin homolog (PTEN); and administering a treatment for DCIS to the subject if the expression level of both RB and PTEN is decreased relative to a reference level; wherein the treatment is not a lumpectomy. In some embodiments, the treatment can be selected from the group consisting of: radiation; chemotherapy; tamoxifen; mastectomy; and radical mastectomy.

In some embodiments, the reference level of expression of (a) retinoblastoma 1 (RB) and (b) phosphatase and tensin homolog (PTEN) can be the level of expression in the breast tissue of a healthy subject with no signs or symptoms of DCIS or any other form of breast cancer. For example, a normal healthy subject can be one with no perceptible calcifications or microcalcifications in their breast tissue as examined by mammography. In some embodiments, the reference can also be a level of expression of RB and PTEN in a control sample, a pooled sample of control individuals, or a numeric value or range of values based on the same. In some embodiments, the reference can also be a level of expression of RB and PTEN in a tissue sample taken from undiseased breast tissue of the subject, e.g. from the contralateral breast. In some embodiments, the reference level of expression of (a) retinoblastoma 1 (RB) and (b) phosphatase and tensin homolog (PTEN) can be the level of expression in non-cancerous tissue of the subject surrounding the ductal carcinoma in situ (DCIS) cells (e.g. the non-cancerous tissue surrounding a DCIS tumor or the non-cancerous tissue surrounding cells with a DCIS phenotype). In certain embodiments, wherein the progression of DCIS in a subject is to be monitored over time, the reference can also be a level of expression of RB and PTEN in a subject's test sample comprising at least one DCIS cell which was taken from breast tissue of the subject at an earlier date (e.g. if “watchful waiting” is selected as a treatment option).

The level of expression of RB or PTEN can be the level of a gene expression product for that gene (e.g. an mRNA or polypeptide). In some embodiments, the same type of gene expression product of each gene is detected. In some embodiments, different types of gene expression products of each gene are detected. In some embodiments, the same assay can be used to detect at least one gene expression product for each gene. In some embodiments, different assays can be used to detect at least one gene expression product for each gene. In some embodiments, the expression levels of both (a) retinoblastoma 1 (RB) and (b) phosphatase and tensin homolog (PTEN) can be normalized relative to the expression level of one or more reference genes, e.g. a housekeeping gene.

In some embodiments, the expression level of RB and PTEN can be determined to be lower than the respective reference levels if the expression of RB and PTEN are statistically significantly lower than the respective reference levels. In some embodiments, the expression level of RB and PTEN can be determined to be lower than the respective reference levels if the expression of RB and PTEN are 50% or less than the respective reference levels, e.g. the levels are 50% or less of the respective reference levels, the levels are 40% or less of respective reference levels, the levels are 30% or less of respective reference levels, the levels are 20% or less of respective reference levels, the levels are 10% or less of respective reference levels, or the levels are 5% or less of respective reference levels. In some embodiments, the subject can be, e.g, at higher risk for experiencing an ipsilateral breast event and/or an ipsilateral breast event which will progress to invasive breast cancer or the subject can be in need of a treatment other than lumpectomy if the expression levels of both (a) retinoblastoma 1 (RB) and (b) phosphatase and tensin homolog (PTEN) are decreased by at least 50% in the test sample relative to the reference level, e.g. the levels are 50% or less of a reference level.

In some embodiments, the expression level of both (a) retinoblastoma 1 (RB) and (b) phosphatase and tensin homolog (PTEN) is the level of mRNA transcript expression product of each gene. Such molecules can be isolated, derived, or amplified from a biological sample, such as a DCIS biopsy or lumpectomy. Assays for detecting mRNA transcripts are well known in the art and include, but are not limited to, PCR procedures, RT-PCR, Northern blot analysis, RNAse protection assay, microarray analysis, hybridization methods etc. In some embodiments, mRNA transcript expression product levels are assayed using reverse transcription polymerase chain reaction (RT-PCR).

The nucleic acid sequences of RB and PTEN have been assigned NCBI accession numbers for different species such as human, mouse and rat. In particular, the NCBI accession numbers for the nucleic acid sequences of the human RB and PTEN expression products are included herein (SEQ ID NOs 001 and 003, respectively). Accordingly, a skilled artisan can design appropriate primers based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the expression level of both (a) retinoblastoma 1 (RB) and (b) phosphatase and tensin homolog (PTEN) is the level of polypeptide expression product of each gene. Assays for detecting polypeptides are well known in the art and include, but are not limited to, ELISA (enzyme linked immunosorbent assay), western blot, immunoprecipitation, immunohistochemistry, and immunofluorescence using detection reagents such as an antibody or protein binding agent. Alternatively, a peptide can be detected in a subject by introducing into the subject a labeled anti-peptide antibody and other types of detection agent. For example, the antibody can be labeled with a radioactive marker whose presence and location in the subject is detected by standard imaging techniques.

Antibodies specific for RB and PTEN are commercially available, (e.g. Cat. Nos. ab24 and ab31392 respectively; Abcam; Cambrige, Mass.) and can be used for the purposes of the methods and assays described herein to measure protein expression levels. Alternatively, since the amino acid sequences for RB and PTEN have been assigned NCBI accession numbers for different species such as human, mouse and rat (e g human sequences are provided as SEQ ID NOs. 002 and 004, respectively) and are publically available at NCBI website, one of skill in the art can raise their own antibodies against these proteins of interest for the methods and assays described herein.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that will undergo a biochemical reaction, and thereby experience, e.g. a change in color, upon encountering the targeted molecules or upon treatment with a chemical agent. In some instances, signal amplification can be integrated into the particular protocol, wherein a secondary antibody, that includes the marker signal or marker activity (e.g. an enzyme activity), follows the application of a primary specific antibody. In some embodiments, an assay as described herein can be performed according to the following steps: a) obtaining a test sample comprising ductal carcinoma in situ (DCIS) cells from the breast tissue of a subject by lumpectomy or biopsy; b) contacting at least one portion of the sample with a primary anti-retinoblastoma 1 (RB) antibody and at least one portion of the sample with a primary anti-phosphatase and tensin homolog (PTEN) antibody; c) washing the sample to remove excess unbound primary antibody; and d) detecting the presence or intensity of a detectable signal; wherein a decrease in the expression level of both RB and PTEN, indicated by the level of the detectable signal, relative to a reference level indicates the subject has a higher risk of experiencing an ipsilateral breast event and/or an ipsilateral breast event which will progress to invasive breast cancer or that the subject is in need of a treatment other than lumpectomy. In some embodiments, the primary antibody is detectably labeled or capable of generating a detectable signal. In some embodiments, an assay as described herein can be performed according to the following steps: a) obtaining a test sample comprising a ductal carcinoma in situ (DCIS) cell from the breast tissue of a subject by lumpectomy or biopsy; b) contacting at least one portion of the sample with a primary anti-retinoblastoma 1 (RB) antibody and at least one portion of the sample with a primary anti-phosphatase and tensin homolog (PTEN) antibody; c) washing the sample to remove excess unbound primary antibody; d) contacting the sample with a secondary antibody having a detectable label or capable of generating a detectable signal; and e) washing the sample to remove excess unbound secondary antibody, and f) detecting the presence or intensity of a detectable signal; wherein a decrease in the expression level of both RB and PTEN, indicated by the level of the detectable signal, relative to a reference level indicates the subject has a higher risk of experiencing an ipsilateral breast event and/or an ipsilateral breast event which will progress to invasive breast cancer or that the subject is in need of a treatment other than lumpectomy.

In some embodiments, one portion of the sample can be contacted with both a primary anti-retinoblastoma 1 (RB) antibody and a primary anti-phosphatase and tensin homolog (PTEN) antibody. In some embodiments, separate portions of the sample can be contacted with a primary anti-retinoblastoma 1 (RB) antibody and a primary anti-phosphatase and tensin homolog (PTEN) antibody.

In some embodiments, multiple antibodies can be used, each of which comprises a different marker or label such that the signal from at least two antibodies can be detected simultaneously in the same sample. In some embodiments, two primary antibodies and two secondary antibodies can be used. In some embodiments, the secondary antibodies can recognize different epitopes, such that each secondary antibody specifically binds to only one of the primary antibodies.

In some embodiments, the methods and assays described herein include (a) transforming gene expression products of RB and PTEN into detectable gene targets; (b) measuring the amount of the detectable gene targets; and (c) comparing the amount of each detectable gene target to an amount of a reference, wherein if the amount of the detectable gene target is statistically lower than that of the amount of the reference level, e.g., the subject is identified as having a higher risk of experiencing an ipsilateral breast event and/or an ipsilateral breast event which will progress to invasive breast cancer. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample. A biological/chemical transformation can involve at least one enzyme and/or a chemical reagent in a reaction. For example, a DNA sample can be digested into fragments by one or more restriction enzymes, or an exogenous molecule can be attached to a fragmented DNA sample with a ligase. In some embodiments, a DNA sample can undergo enzymatic replication, e.g., by polymerase chain reaction (PCR).

In some embodiments, an assay described herein can comprise detecting the expression level of no more than 20 genes other than RB and PTEN. In some embodiments, an assay described herein can comprise detecting the expression level of no more than 10 genes other than RB and PTEN.

In some embodiments, the level of risk can be expressed in terms of a 1) hazard ratio, 2) the likelihood of experiencing an ipsilateral breast event, and/or 3) the likelihood of experiencing an ipsilateral breast event which will progress to invasive breast cancer.

In some embodiments, a subject determined to have RB and PTEN expression levels in a test sample which are less than 50% of a reference expression level (e.g. 45% or less, 40% or less, 30% or less, 20% or less, or 10% or less of the reference) can be more likely to experience an ipsilateral breast event or to experience an ipsilateral breast event which will progress to invasive breast cancer as compared to a reference.

In some embodiments, a subject determined to have RB and PTEN expression levels in a test sample which are less than 50% of a reference expression level can be at least 2 times more likely to experience an ipsilateral breast event as compared to the reference, e.g. at least 2 times more likely, at least 3 times more likely, at least 4 more likely, or at least 5 times more likely. In some embodiments, a subject determined to have RB and PTEN expression levels in a test sample which are less than 50% of a reference expression level can be about 3.39 times more likely to experience an ipsilateral breast event as compared to the reference.

In some embodiments, a subject determined to have RB and PTEN expression levels in a test sample which are less than 50% of a reference expression level can be at least 2 times more likely to experience an ipsilateral breast event which will progress to invasive breast cancer as compared to the reference, e.g. at least 2 times more likely, at least 3 times more likely, at least 4 times more likely, at least 5 time more likely, at least 6 times more likely, at least 7 times more likely, or at least 8 times more likely. In some embodiments, a subject determined to have RB and PTEN expression levels in a test sample which are less than 50% of a reference expression level can be about 6.1 times more likely to experience an ipsilateral breast event which will progress to invasive breast cancer as compared to the reference.

In some embodiments, a subject determined to have RB and PTEN expression levels in a test sample which are less than 50% of a reference expression level can have about an 18% chance of experiencing an ipsilateral breast event within 1 year, about a 36% chance of experiencing an ipsilateral breast event within 2 years, about a 61% chance of experiencing an ipsilateral breast event within 5 years, and/or about a 71% chance of experiencing an ipsilateral breast event within 10 years.

In some embodiments, a subject determined to have RB and PTEN expression levels in a test sample which are less than 50% of a reference expression level can have about an 8% chance of experiencing an ipsilateral breast event which will progress to invasive breast cancer within 1 year, about a 16% chance of experiencing an ipsilateral breast event which will progress to invasive breast cancer within 2 years, about a 38% chance of experiencing an ipsilateral breast event which will progress to invasive breast cancer within 5 years, and/or about a 53% chance of experiencing an ipsilateral breast event which will progress to invasive breast cancer within 10 years.

In some embodiments, the assays and methods described herein can comprise determining a likelihood of a subject experiencing an ipsilateral breast event and/or an ipsilateral breast event which progresses to invasive cancer based upon the expression level of RB and PTEN in a test sample obtained from that subject as compared to known likelihoods. Such likelihoods can be calculated for groups of subjects with varying levels of expression of RB and PTEN (e.g. 40% of a reference level, or 20% of a reference level) and those values used to determine the relative risk of a subsequent subject. In some embodiments, the values can be calculated using the Kaplan-Meier method. In some embodiments, the assays and methods described herein can comprise determining a hazard ratio for a subject based upon the expression level of RB and PTEN in a test sample obtained from that subject as compared to known hazard values. Hazard ratios can be calculated for groups of subjects with varying levels of expression of RB and PTEN (e.g. 40% of a reference level, or 20% of a reference level) and those hazard ratios used to determine the relative risk of a subsequent subject. In some embodiments, the hazard ratio can be calculated using the Cox proportional-hazards regression model. The calculations described above can be performed using statistical software, e.g. SAS™ software (version 9.2; SAS Institute, Cary, N.C.).

In some embodiments, any of the assays or methods described herein can further comprise creating a report based on the expression level of both (a) retinoblastoma 1 (RB) and (b) phosphatase and tensin homolog (PTEN). Such reports can comprise. e.g., expression levels of at least RB and PTEN, normalized expression levels of at least RB and PTEN, the expression level of at least RB and PTEN as compared to reference levels, the risk of the subject experiencing an ipislateral breast event and/or an ipsilateral breast event which will progress to invasive breast cancer, and/or whether the subject is in need of treatment other than lumpectomy.

### Subjects

In some embodiments, a test sample can be from a subject having, at risk of having, or diagnosed as having ductal carcinoma in situ (DCIS). In some embodiments, a subject can be exhibiting a sign or symptom of DCIS. The diagnosis of DCIS is well understood in the art and can be made, e.g., by taking into account the presence of calcifications (or microcalcifications) in the ducts detected by a mammogram. Ultrasound and MRI imaging technologies can also be useful in order to examine lumps or calcifications via a noninvasive means. Breast tissue biopsies are often used to permit or confirm a diagnosis of DCIS. Examples of biopsy techniques include a core needle biopsy, a stereotactic biopsy, incisional biopsy, and a surgical biopsy (e.g. wide local excision or lumpectomy). In some embodiments, a lumpectomy can also be a treatment for DCIS, e.g. by removing the cancerous cells. Biopsy samples can be examined for signs of abnormal cells or growth, e.g. ductal hyperplasia, atypical ductal hyperplasia, too many cells in the duct, cells with abnormal phenotypes in the duct, microinvasion (i.e. a small number of cancer cells beginning to penetrate the ductal wall), papillae of cells in the duct, cribiform growth of cells in the duct, a duct completely occupied by cancerous cells, and/or comedo necrosis (i.e. an area of dead cells in the population of cancerous cells). Biopsy samples can also be examined to determined the presence of hormone receptors (e.g. the estrogen receptor and the progesterone receptor), which can offer information regarding the severity of the DCIS diagnosis and/or appropriate treatment regimens. In some embodiments, the subject is a human subject. In some embodiments, the subject is a female subject.

### Test Samples

Provided herein are methods, assays and systems relating to determining the expression level of RB and PTEN in DCIS cells of a subject. The term “test sample” as used herein denotes a sample taken or isolated from a biological organism, e.g., breast tissue biopsy sample, cell lysate, a homogenate of a tissue sample from a subject or a fluid sample from a subject. Exemplary biological samples include, but are not limited to, breast tissue biopsies, or tissue removed via lumpectomy, etc. The term also includes a mixture of the above-mentioned samples. The term “test sample” also includes untreated or pretreated (or pre-processed) biological samples. A test sample can contain cells from subject, but the term can also refer to non-cellular biological material, such as non-cellular fractions that can be used to measure gene expression levels. In some embodiments, the sample is from a resection, biopsy, or core needle biopsy. In addition, fine needle aspirate samples can be used. Samples can also include either paraffin-embedded or frozen tissue.

In some embodiments, the test sample can comprise a ductal carcinoma in situ (DCIS) carcinoma or a portion thereof. In some embodiments, the test sample can comprise at least one DCIS cell, i.e. at least one cell displaying a DCIS phenotype or which, in vivo, was part of a cell mass displaying a DCIS phenotype.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, sonication, homogenization, lysis, thawing, amplification, purification, restriction enzyme digestion ligation and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample. The skilled artisan is well aware of methods and processes appropriate for pre-processing of biological samples required for determination of expression of gene products as described herein.

### Treatment

When DCIS is diagnosed, subjects typically undergo lumpectomy to remove all identified “lumps” of DCIS along with “clear margins” (i.e. a border of healthy tissue surrounding the cancerous cells. For subjects with a large area of DCIS, a mastectomy or radical mastectomy may be necessary. Subjects can also elect to undergo a mastectomy or radical mastectomy where such procedures are not deemed medically necessary by a physician.

In some embodiments, a subject treated according to the methods described herein, or in need of multiple treatments or aggressive treatment as determined according to the methods described herein can undergo a mastectomy or radical mastectomy. In some embodiments, a subject treated according to the methods described herein, or in need of multiple treatments or aggressive treatment as determined according to the methods described herein can undergo radiation therapy. By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

In some embodiments, a subject treated according to the methods described herein, or in need of multiple treatments or aggressive treatment as determined according to the methods described herein can be administered a chemotherapeutic agent. In some embodiments, the chemotherapeutic agent can be tamoxifen. Further examples of chemotherapeutic agents include, but are not limited to, gemcitabine, cisplastin, paclitaxel, carboplatin, bortezomib, AMG479, vorinostat, rituximab, temozolomide, rapamycin, ABT-737, PI-103, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN@ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In some embodiments, combinations of any of the above treatments can be administered.

### Computer Systems

In some embodiments of the assays and/or methods described herein, the assay/method comprises or consists essentially of a system for determining (e.g. transforming and measuring) the expression level of RB and PTEN as described herein and comparing them to a reference expression level. If the comparison system, which can be a computer implemented system, indicates that the amount of the measured gene expression product is statistically different from that of the reference amount, the subject from which the sample is collected can be identified as, e.g. having an increased risk of experiencing an ipsilateral breast event or as having a higher risk carcinoma.

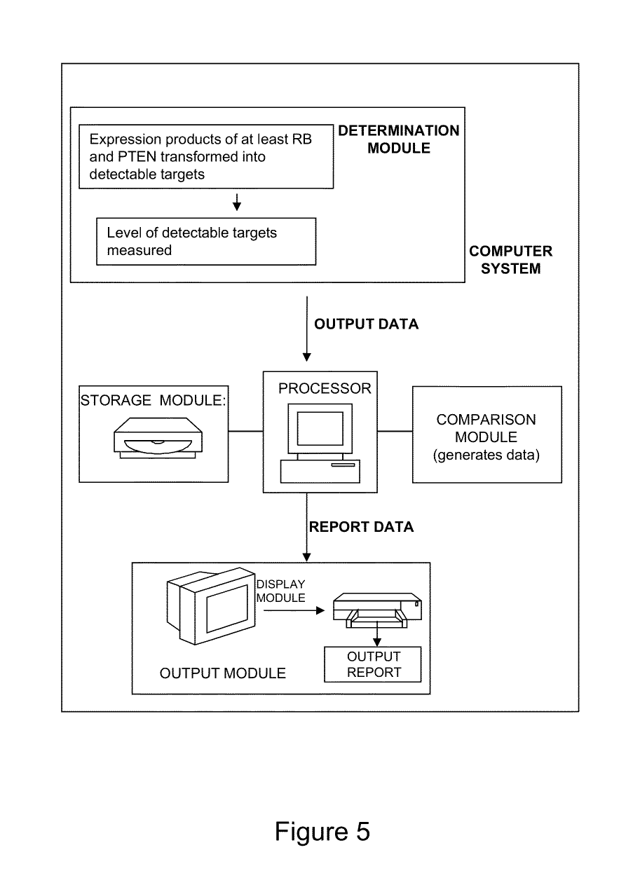

In one embodiment, provided herein is a system comprising: (a) at least one memory containing at least one computer program adapted to control the operation of the computer system to implement a method that includes (i) a determination module configured to identify and detect at the expression level of RB and PTEN in a test sample obtained from a subject; (ii) a storage module configured to store output data from the determination module; (iii) a computing module adapted to identify from the output data whether the level of expression of RB and PTEN in the test sample obtained from the subject is lower, by a statistically significant amount, than a reference expression level, and (iv) a display module for displaying whether RB and PTEN expression levels are lower in the test sample as compared to a reference level and/or displaying the relative expression levels of RB and PTEN and (b) at least one processor for executing the computer program (see FIG. 5).

Embodiments can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. Computer-readable storage medium do not include a signal.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments can include at minimum a determination module, a storage module, a computing module, and a display module. The functional modules can be executed on one, or multiple, computers, or by using one, or multiple, computer networks. The determination module has computer executable instructions to provide e.g., levels of expression products etc in computer readable form.

The determination module can comprise any system for detecting a signal elicited from the expression products of RB and PTEN in a biological sample. In some embodiments, such systems can include an instrument, e.g., STEPONEPLUS REAL-TIME™ PCR systems (Applied Biosystems; Carlsbad, Calif.) for quantitative RT-PCR. In another embodiment, the determination module can comprise multiple units for different functions, such as amplification and hybridization. In one embodiment, the determination module can be configured to perform the quantitative RT-PCR methods including amplification, detection, and analysis. In some embodiments, such systems can include an instrument, e.g., the Cell Biosciences NANOPRO 1000™ System (Protein Simple; Santa Clara, Calif.) for quantitative measurement of peptides and/or proteins.

In some embodiments, the determination module can be further configured to identify and detect the presence of at least one additional gene expression product.

The information determined in the determination system can be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as expression levels of RB and PTEN in subjects who do not have signs, symptoms, or markers of DCIS or another breast cancer. In certain embodiments, the storage module stores the reference information such as expression levels of RB and PTEN in a sample of healthy breast tissue obtained from the subject.

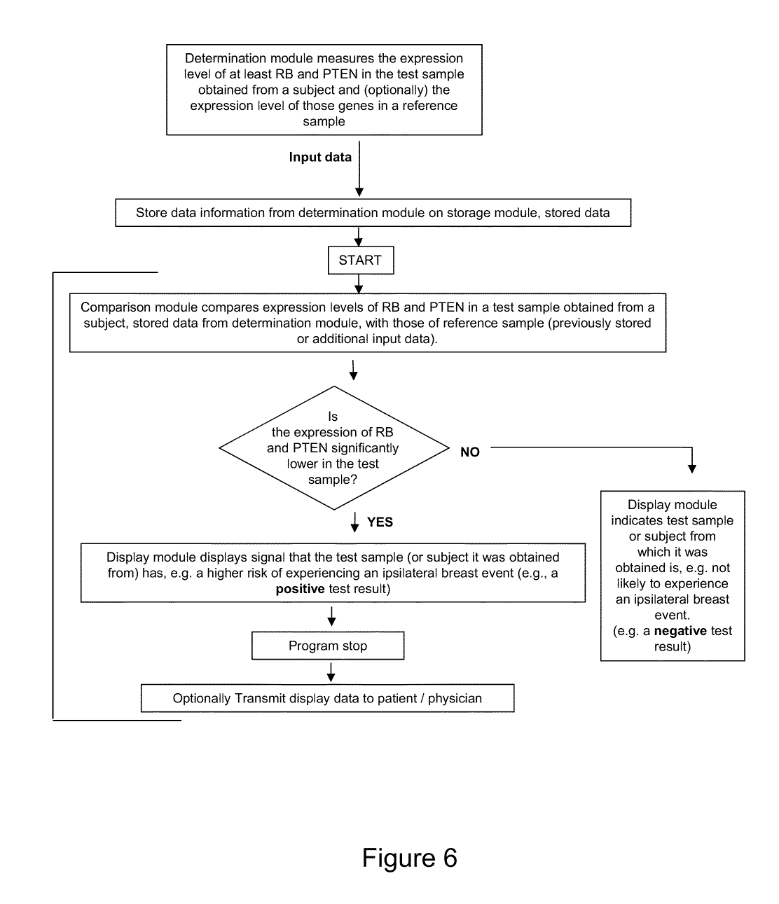

The “computing module” can use a variety of available software programs and formats for computing the relative expression level of RB and PTEN. Such algorithms are well established in the art. A skilled artisan is readily able to determine the appropriate algorithms based on the size and quality of the sample and type of data. The data analysis can be implemented in the computing module. In one embodiment, the computing module further comprises a comparison module, which compares the expression level of RB and PTEN in the test sample obtained from a subject as described herein with the reference expression level of those genes (FIG. 6). By way of example, when the expression level of RB in the test sample obtained from a subject is measured, a comparison module can compare or match the output data, e.g. with the reference expression level of RB in a reference sample. In certain embodiments, the reference expression level can have been pre-stored in the storage module. During the comparison or matching process, the comparison module can determine whether the expression level in the test sample obtained from a subject is lower than the reference expression level to a statistically significant degree. In various embodiments, the comparison module can be configured using existing commercially-available or freely-available software for comparison purpose, and may be optimized for particular data comparisons that are conducted.

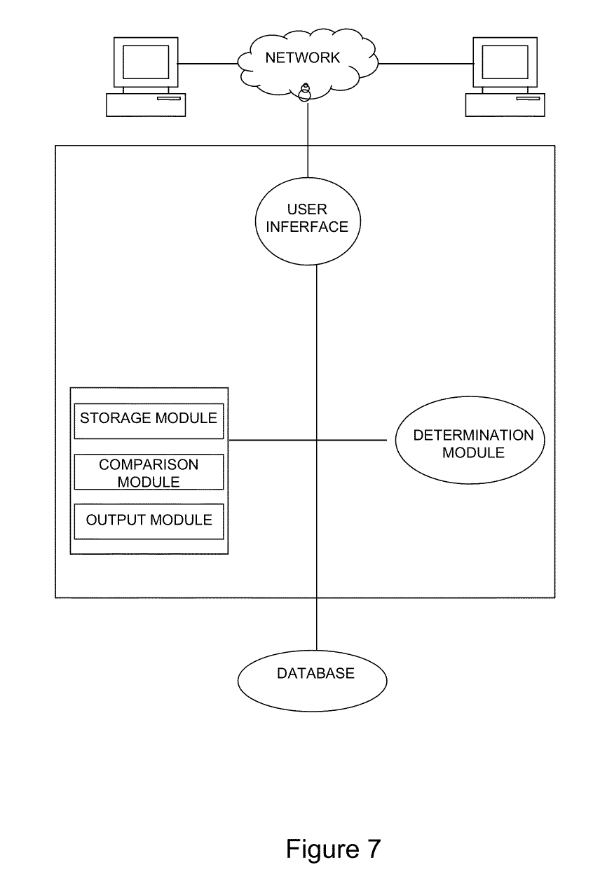

The computing and/or comparison module, or any other module, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware. as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment, users can directly access data (via Hypertext links for example) residing on Internet databases using a HTML interface provided by Web browsers and Web servers (FIG. 7).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, described herein is a computer system for determining the risk of a subject experiencing an ipsilateral breast event, the system comprising: a measuring module configured to measure the expression level of (a) retinoblastoma 1 (RB) and (b) phosphatase and tensin homolog (PTEN) in a test sample obtained from a subject; a storage module configured to store output data from the determination module; a comparison module adapted to compare the data stored on the storage module with a reference level, and to provide a retrieved content, and a display module for displaying whether RB and PTEN expression products have a statistically significant decrease in expression level in the test sample obtained from a subject as compared to the reference expression level and/or displaying the relative expression levels of the marker gene products. In some embodiments, the measuring module can measure the intensity of a detectable signal from an immunoassay indicating the presence or level of (a) retinoblastoma 1 (RB) and (b) phosphatase and tensin homolog (PTEN) polypeptides in the test sample. In some embodiments, the measuring module can measure the intensity of a detectable signal from a RT-PCR assay indicating the presence or level of (a) retinoblastoma 1 (RB) and (b) phosphatase and tensin homolog (PTEN) RNA transcripts in the test sample. In some embodiments, the reference expression level can be the level of (a) retinoblastoma 1 (RB) and (b) phosphatase and tensin homolog (PTEN) in the breast tissue of a population of healthy subjects. In some embodiments, the reference expression level can be the level of (a) retinoblastoma 1 (RB) and (b) phosphatase and tensin homolog (PTEN) in the healthy breast tissue of the subject with ductal carcinoma in situ (DCIS).

In some embodiments, the content displayed on the display module can be the relative expression levels of RB and PTEN in the test sample obtained from a subject as compared to a reference expression level. In certain embodiments, the content displayed on the display module can indicate whether RB and PTEN were found to have a statistically significantly lower expression in the test sample obtained from a subject as compared to a reference expression level. In certain embodiments, the content displayed on the display module can indicate the degree to which RB and PTEN were found to have a statistically significantly lower expression level in the test sample obtained from a subject as compared to a reference expression level. In certain embodiments, the content displayed on the display module can indicate whether the subject has an increased risk of experiencing an ipsilateral breast event and/or an ipsilateral breast event which will progress to invasive breast cancer. In certain embodiments, the content displayed on the display module can indicate whether the subject is in need of a multiple treatments for DCIS. In certain embodiments, the content displayed on the display module can indicate whether the subject has a high risk DCIS carcinoma. In some embodiments, the content displayed on the display module can be a numerical value indicating one of these risks or probabilities. In such embodiments, the probability can be expressed in percentages or a fraction. For example, higher percentage or a fraction closer to 1 indicates a higher likelihood of a subject experiencing an ipsilateral breast event. In some embodiments, the content displayed on the display module can be single word or phrases to qualitatively indicate a risk or probability. For example, a word “unlikely” can be used to indicate a lower risk for experiencing an ipsilateral breast event, while “likely” can be used to indicate a high risk for experiencing an ipsilateral breast event.

In one embodiment, the content based on the computing and/or comparison result is displayed on a computer monitor. In one embodiment, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the technology relating to determining the expression level of RB and PTEN, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Devices/Kits

Provided herein are kits and devices for practicing the assays and methods described herein.

In some embodiments, described herein is a device for measuring the presence or level of (i) retinoblastoma 1 (RB) and (ii) phosphatase and tensin homolog (PTEN) expression in a test sample from a subject comprising: (a) at least a RB-specific antibody or antigen-binding portion thereof and a PTEN-specific antibody or antigen-binding portion thereof; and (b) at least one solid support, wherein the antibodies or antigen-binding portions thereof of step a are deposited on the support. In some embodiments, the device can perform an assay in which an antibody-protein or antibody-peptide complex is formed. In some embodiments, the solid support can be in the format of a dipstick, a microfluidic chip, a multi-well plate or a cartridge. The kits or devices can employ immuno-based lateral flow technology to produce a signal.

In some embodiments, described herein is a kit comprising: a device as described in the preceding paragraph; and at least a detection antibody. In some embodiments, the detection antibody can be specific for (a) retinoblastoma 1 (RB) or (b) phosphatase and tensin homolog (PTEN). In some embodiments, the detection antibody can be detectably labeled. In some embodiments, the kit can further comprise at least an agent for producing a detectable signal from the detection antibody.

In some embodiments, the kit or device can comprise a reference, e.g. a reference sample or reference signal. In some embodiments, the reference can comprise a sample of breast tissue from a healthy subject. In some embodiments, the reference can comprise purified, isolated, recombinant, and/or synthetic, RB and/or PTEN or fragments or epitopes thereof present a concentration equivalent to that found in the breast tissue of a healthy subject. In some embodiments, the reference can be a signal equivalent to the signal that would be obtained when the particular assay is performed on breast tissue of a healthy subject.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. An assay comprising:
   subjecting a test sample from a subject to at least one analysis to determine the level of expression of both (a) retinoblastoma 1 (RB) and (b) phosphatase and tensin homolog (PTEN);
   wherein a decrease in expression of both RB and PTEN relative to a reference level indicates the subject has a higher risk of experiencing an ipsilateral breast event.
2. The assay of paragraph 1, wherein a decrease in expression of both RB and PTEN relative to a reference level indicates the subject has a higher risk of experiencing an ipsilateral breast event which will progress to invasive breast cancer.
3. The assay of any of paragraphs 1-2, wherein the subject has ductal carcinoma in situ (DCIS).
4. The assay of any of paragraphs 1-3, wherein the expression level of both (a) retinoblastoma 1 (RB) and (b) phosphatase and tensin homolog (PTEN) is the level of RNA transcript expression product of each gene.
5. The assay of paragraph 4, wherein the RNA transcript expression product levels are assayed using reverse transcription polymerase chain reaction (RT-PCR).
6. The assay of any of paragraphs 1-3, wherein the expression level of both (a) retinoblastoma 1 (RB) and (b) phosphatase and tensin homolog (PTEN) is the level of polypeptide expression product of each gene.
7. The assay of paragraph 6, wherein the polypeptide expression product levels are assayed using immunohistological staining
8. An assay comprising:
   (a) obtaining a test sample comprising a ductal carcinoma in situ (DCIS) cell from the breast tissue of a subject by lumpectomy or biopsy;
   (b) contacting at least one portion of the sample with a primary anti-retinoblastoma 1 (RB) antibody and at least one portion of the sample with a primary anti-phosphatase and tensin homolog (PTEN) antibody;
   (c) washing the sample to remove excess unbound primary antibody; and
   (d) detecting the presence or intensity of a detectable signal;
   wherein a decrease in the expression level of both RB and PTEN, indicated by the level of the detectable signal, relative to a reference level indicates the subject has a higher risk of experiencing an ipsilateral breast event.
9. The assay of paragraph 8, wherein a decrease in expression of both RB and PTEN relative to a reference level indicates the subject has a higher risk of experiencing an ipsilateral breast event which will progress to invasive breast cancer.
10. The assay of any of paragraphs 8-9, wherein the primary antibody is detectably labeled or capable of generating a detectable signal.
11. The assay of any of paragraphs 8-9, wherein, between steps (c) and (d), the assay further comprises:
   contacting the sample with a secondary antibody having a detectable label or capable of generating a detectable signal; and
   washing the sample to remove excess unbound secondary antibody.
12. The assay of any of paragraphs 8-11, wherein one portion of the sample is contacted with both a primary anti-retinoblastoma 1 (RB) antibody and a primary anti-phosphatase and tensin homolog (PTEN) antibody.
13. The assay of any of paragraphs 8-11, wherein separate portions of the sample are contacted with a primary anti-retinoblastoma 1 (RB) antibody and a primary anti-phosphatase and tensin homolog (PTEN) antibody.
14. The assay of any of paragraphs 1-13, wherein the subject is at higher risk for experiencing an ipsilateral breast event if the expression levels of both (a) retinoblastoma 1 (RB) and (b) phosphatase and tensin homolog (PTEN) are decreased by at least 50% in the test sample relative to the reference level.
15. The assay of any of paragraphs 1-14, wherein the risk of recurrence is expressed in terms of a hazard ratio, the likelihood of experiencing an ipsilateral breast event, and/or the likelihood of experiencing an ipsilateral breast event which will progress to invasive breast cancer.
16. An assay to determine if a subject with ductal carcinoma in situ (DCIS) is in need of treatment with therapy other than a lumpectomy, the assay comprising:
   subjecting a test sample of a subject to at least one analysis to determine the level of expression of both (a) retinoblastoma 1 (RB) and (b) phosphatase and tensin homolog (PTEN);
   wherein a decrease in expression of both RB and PTEN relative to a reference level indicates the subject is in need of treatment with a therapy other than a lumpectomy.
17. The assay of paragraph 16, wherein the treatment other than a lumpectomy is selected from the group consisting of:
   radiation; chemotherapy; tamoxifen; mastectomy; and radical mastectomy.
18. The assay of any of paragraphs 16-17, wherein
   wherein the individual is in need of treatment with a therapy other than a lumpectomy if the expression levels of both (a) retinoblastoma 1 (RB) and (b) phosphatase and tensin homolog (PTEN) are decreased by at least 50% in the test sample relative to the reference level.
19. The assay of any of paragraphs 1-18, wherein the expression level of both (a) retinoblastoma 1 (RB) and (b) phosphatase and tensin homolog (PTEN) are normalized relative to the expression level of one or more reference genes.
20. The assay of any of paragraphs 1-19, wherein the test sample comprises a ductal carcinoma in situ (DCIS) cell.
21. The assay of any of paragraphs 1-20, wherein the test sample is obtained by performing a lumpectomy or biopsy on the subject.
22. The assay of any of paragraphs 1-21, wherein the reference level of expression of (a) retinoblastoma 1 (RB) and (b) phosphatase and tensin homolog (PTEN) is the level of expression in non-cancerous tissue of the subject surrounding a ductal carcinoma in situ (DCIS) cell.
23. The assay of any of paragraphs 1-22, wherein the reference level of expression of (a) retinoblastoma 1 (RB) and (b) phosphatase and tensin homolog (PTEN) is the level of expression in the breast tissue of a healthy subject.
24. The assay of any of paragraphs 1-23, wherein the expression level of no more than 20 other genes is determined
25. The assay of any of paragraphs 1-24, wherein the expression level of no more than 10 other genes is determined
26. The assay of any of paragraphs 1-25, wherein the subject is a human.
27. The assay of any of paragraphs 1-26, further comprising creating a report based on the expression level of both (a) retinoblastoma 1 (RB) and (b) phosphatase and tensin homolog (PTEN).
28. A method of administering a treatment for ductal carcinoma in situ (DCIS) to a subject, the method comprising:
   subjecting a test sample of a subject to at least one analysis to determine the level of expression of both (a) retinoblastoma 1 (RB) and (b) phosphatase and tensin homolog (PTEN); and
   administering a treatment for DCIS to the subject if the expression level of both RB and PTEN is decreased relative to a reference level;
   wherein the treatment is not a lumpectomy.
29. The method of paragraph 28, wherein the aggressive treatment for ductal carcinoma in situ (DCIS) is selected from the group consisting of:
   radiation; chemotherapy; tamoxifen; mastectomy; and radical mastectomy.
30. A method of identifying a subject with ductal carcinoma in situ (DCIS) who is in need of multiple treatments, the method comprising:
   subjecting a test sample of a subject to at least one analysis to determine the level of expression of both (a) retinoblastoma 1 (RB) and (b) phosphatase and tensin homolog (PTEN); and
   wherein the subject is identified as needing multiple treatments for DCIS if the expression level of both RB and PTEN is decreased relative to a reference level.
31. The method of paragraph 30, wherein the multiple treatments for ductal carcinoma in situ (DCIS) comprise:
   (a) lumpectomy; and
   (b) at least one further treatment selected from the group consisting of:
   radiation; chemotherapy; tamoxifen; mastectomy; and radical mastectomy.
32. A method of classifying a ductal carcinoma in situ (DCIS) carcinoma, the method comprising:
   subjecting a test sample of a subject to at least one analysis to determine the level of expression of both (a) retinoblastoma 1 (RB) and (b) phosphatase and tensin homolog (PTEN); and
   (a) classifying the DCIS as a high risk carcinoma if the expression level of both RB and PTEN is decreased relative to a reference level;
   (b) classifying the DCIS as a low risk carcinoma if the expression level of both RB and PTEN is not decreased relative to a reference level.
33. The method of any of paragraphs 28-32, wherein the expression level of both (a) retinoblastoma 1 (RB) and (b) phosphatase and tensin homolog (PTEN) is the level of RNA transcript expression product of each gene.
34. The method of paragraph 33, wherein the RNA transcript expression product levels are assayed using reverse transcription polymerase chain reaction (RT-PCR).
35. The method of any of paragraphs 28-32, wherein the expression level of both (a) retinoblastoma 1 (RB) and (b) phosphatase and tensin homolog (PTEN) is the level of polypeptide expression product of each gene.
36. The method of paragraph 35, wherein the polypeptide expression product levels are assayed using immunohistological staining
37. The method of any of paragraphs 28-36, wherein the expression level of both (a) retinoblastoma 1 (RB) and (b) phosphatase and tensin homolog (PTEN) are normalized relative to the expression level of one or more reference genes.
38. The method of any of paragraphs 28-37, wherein the test sample comprises a ductal carcinoma in situ (DCIS) cell.
39. The method of any of paragraphs 28-38, wherein the test sample is obtained by performing a lumpectomy or biopsy on the subject.
40. The method of any of paragraphs 28-39, wherein the reference level of expression of (a) retinoblastoma 1 (RB) and (b) phosphatase and tensin homolog (PTEN) is the level of expression in non-cancerous tissue of the subject surrounding a DCIS cell.
41. The method of any of paragraphs 28-40, wherein the reference level of expression of (a) retinoblastoma 1 (RB) and (b) phosphatase and tensin homolog (PTEN) is the level of expression in the breast tissue of a healthy subject.
42. The method of any of paragraphs 28-41, wherein the expression level of no more than 20 other genes is determined.
43. The method of any of paragraphs 28-42, wherein the expression level of no more than 10 other genes is determined.
44. The method of any of paragraphs 28-43, wherein the subject is a human.
45. A method of determining whether a subject is at risk of experiencing a ipsilateral breast event, the method comprising:
   determining the level of expression of both (a) retinoblastoma 1 (RB) and (b) phosphatase and tensin homolog (PTEN) in a test sample obtained from a ductal carcinoma in situ (DCIS) cell from a subject;
   wherein a decrease in expression of both RB and PTEN relative to a reference level indicates the subject has a higher risk experiencing an ipsilateral breast event.
46. The assay of paragraph 45, wherein a decrease in expression of both RB and PTEN relative to a reference level indicates the subject has a higher risk of experiencing an ipsilateral breast event which will progress to invasive breast cancer.
47. The method of any of paragraphs 45-46, wherein the expression level of both (a) retinoblastoma 1 (RB) and (b) phosphatase and tensin homolog (PTEN) is the level of RNA transcript expression product of each gene.
48. The method of paragraph 47, wherein the RNA transcript expression product levels are assayed using reverse transcription polymerase chain reaction (RT-PCR).
49. The method of any of paragraphs 45-46, wherein the expression level of both (a) retinoblastoma 1 (RB) and (b) phosphatase and tensin homolog (PTEN) is the level of polypeptide expression product of each gene.
50. The method of paragraph 49, wherein the polypeptide expression product levels are assayed using immunohistological staining
51. The method of any of paragraphs 45-50, wherein the expression level of both (a) retinoblastoma 1 (RB) and (b) phosphatase and tensin homolog (PTEN) are normalized relative to the expression level of one or more reference genes.
52. The method of any of paragraphs 45-51, wherein the subject is at higher risk for experiencing an ipsilateral breast event if the expression levels of both (a) retinoblastoma 1 (RB) and (b) phosphatase and tensin homolog (PTEN) are decreased by at least 50% in the test sample relative to the reference level.
53. The method of any of paragraphs 45-52, wherein the risk of recurrence is expressed in terms of a hazard ratio, the likelihood of experiencing an ipsilateral breast event, and/or the likelihood of experiencing an ipsilateral breast event which will progress to invasive breast cancer.
54. The method of any of paragraphs 45-53, wherein a subject with a higher risk of experiencing an ipsilateral breast event is indicated to receive a treatment selected from the group consisting of:
   radiation; chemotherapy; tamoxifen; mastectomy; and radical mastectomy.
55. The assay of any of paragraphs 45-54, wherein the reference level of expression of (a) retinoblastoma 1 (RB) and (b) phosphatase and tensin homolog (PTEN) is the level of expression in non-cancerous tissue of the subject surrounding a ductal carcinoma in situ (DCIS) cell.
56. The assay of any of paragraphs 45-55, wherein the reference level of expression of (a) retinoblastoma 1 (RB) and (b) phosphatase and tensin homolog (PTEN) is the level of expression in the breast tissue of a healthy subject.
57. The assay of any of paragraphs 45-56, wherein the expression level of no more than 20 other genes is determined.
58. The assay of any of paragraphs 45-57, wherein the expression level of no more than 10 other genes is determined.
59. The assay of any of paragraphs 45-58, wherein the subject is a human.
60. A computer system for determining the risk of a subject experiencing an ipsilateral breast event, the system comprising:
   a determination module configured to measure the expression level of (a) retinoblastoma 1 (RB) and (b) phosphatase and tensin homolog (PTEN) in a test sample obtained from a subject;
   a storage module configured to store output data from the determination module;
   a comparison module adapted to compare the data stored on the storage module with a reference level, and to provide a retrieved content, and
   a display module for displaying whether RB and PTEN expression products have a statistically significant decrease in expression level in the test sample obtained from a subject as compared to the reference expression level and/or displaying the relative expression levels of the marker gene products.
61. The system of paragraph 60, wherein the measuring module measures the intensity of a detectable signal from an immunoassay indicating the presence or level of (a) retinoblastoma 1 (RB) and (b) phosphatase and tensin homolog (PTEN) polypeptides in the test sample.
62. The system of paragraph 60, wherein the measuring module measures the intensity of a detectable signal from a RT-PCR assay indicating the presence or level of (a) retinoblastoma 1 (RB) and (b) phosphatase and tensin homolog (PTEN) RNA transcripts in the test sample.
63. The system of any of paragraphs 60-62, wherein the reference expression level is the level of (a) retinoblastoma 1 (RB) and (b) phosphatase and tensin homolog (PTEN) in the breast tissue of a population of healthy subjects.
64. The system of any of paragraphs 60-63, wherein the reference expression level is the level of (a) retinoblastoma 1 (RB) and (b) phosphatase and tensin homolog (PTEN) in the healthy breast tissue of the subject with ductal carcinoma in situ (DCIS).
65. The system of any of paragraphs 60-64, wherein if the computing module determines that the expression level of (a) retinoblastoma 1 (RB) and (b) phosphatase and tensin homolog (PTEN) in the test sample obtained from a subject is lower by a statistically significant amount than the reference expression level, the display module displays a signal indicating that the expression levels in the sample obtained from a subject are lower than those of the reference expression level.
66. The system of any of paragraphs 60-65, wherein the signal indicates that the subject has an increased likelihood of experiencing an ipsilateral breast event.
67. The system of any of paragraphs 60-66, wherein the signal indicates that the subject has an increased likelihood of experiencing an ipsilateral breast event which will progress to invasive breast cancer.
68. The system of any of paragraphs 60-67, wherein the signal indicates the subject is in need of aggressive treatment or multiple forms of treatment.
69. The system of any of paragraphs 60-68, wherein the signal indicates the degree to which the expression level of (a) retinoblastoma 1 (RB) and (b) phosphatase and tensin homolog (PTEN) in the sample obtained from a subject vary from the reference expression level.
70. A device for measuring the presence or level of (a) retinoblastoma 1 (RB) and (b) phosphatase and tensin homolog (PTEN) expression in a test sample from a subject comprising:
   (a) at least a RB-specific antibody or antigen-binding portion thereof and a PTEN-specific antibody or antigen-binding portion thereof; and
   (b) at least one solid support, wherein the antibodies or antigen-binding portions thereof of step a are deposited on the support.
71. The device of paragraph 70, wherein the device performs an assay in which an antibody-protein or antibody-peptide complex is formed.
72. The device of any one of paragraphs 70-71, the solid support is in the format of a dipstick, a microfluidic chip, a multi-well plate or a cartridge.
73. The device of any of paragraphs 70-72, further comprising a reference.
74. A kit comprising:
   a device according to any of paragraphs 70-73; and
   at least a detection antibody.
75. The kit of paragraph 74, wherein the detection antibody is specific for (a) retinoblastoma 1 (RB) or (b) phosphatase and tensin homolog (PTEN).
76. The kit of any of paragraphs 74-75, wherein the detection antibody is detectably labeled.
77. The kit of any of paragraphs 74-76, further comprising at least an agent for producing a detectable signal from the detection antibody.

### EXAMPLES

### Example 1

Ductal carcinoma in situ (DCIS) is a nonobligatory precursor to invasive breast cancer (IBC). With increased use of screening mammography to detect occult breast cancer, the incidence of DCIS markedly increased, and it is estimated that 1 million women will be living with this condition by 2020. Left untreated up to 53% DCIS will progress to invasive breast cancer. Unfortunately, DCIS classifications used in clinical practice do not adequately predict risk of DCIS recurrence and progression. Recently a new pathologic grading system was proposed to improve prediction of local recurrence⁵. In this system, DCIS with high nuclear grade, predominantly solid architecture and exhibiting extensive (present in >50% of ducts) comedo-type necrosis had particularly bad prognosis and was associated with recurrence or progression to IBC in less than 10 years. Sanders et al. examined the natural history of untreated, low grade, noncomedo DCIS and showed that 39.3% of these patients developed invasive breast cancer in the same quadrant as the initial biopsy with most events occurring within 10-15 years, but with some as late as 23-42 years⁶. Nearly half of the patients who developed invasive breast cancer died of metastatic disease 1-7 years after diagnosis. The results of that study suggest that a subset of patients with low grade DCIS will develop life-threatening invasive carcinoma. Stratifying these patients using prognostic cancer markers can help to prevent both under and over treatment.

Identification of mechanisms driving invasive progression of DCIS can facilitate development of better prognostic tests for DCIS patients and is an area of active investigation. The 2009 State of Science NIH Conference on Diagnosis and Management of DCIS recommended development of risk stratification tests based on a comprehensive understanding of the clinical, radiological, pathological, and biological factors associated with DCIS. Numerous biomarkers have been investigated for risk stratification of patients with DCIS. Elevated Ki-67 levels, p53 mutations, and HER2 amplification are known to be associated with increased nuclear grade and necrosis which have also been associated with disease recurrence and progression⁷⁻⁹. Cell cycle markers have also been studied including p21, p27, and cyclin D1¹⁰⁻¹². Overexpression of p16ink4a and concomitant elevated proliferative marker Ki67 were observed in DCIS at risk for progressing to invasive breast carcinomas^{8,13}. Despite these studies, no single biomarker has been identified to guide proper and effective therapies. In particular, the role of PTEN in DCIS progression has not been investigated, nor has the combined role of PTEN and RB, either in DCIS progression, or in prognosis of DCIS progression been evaluated.

### Materials and Methods

Patient Selection:

DCIS breast tissue was obtained from the surgical pathology files at Thomas Jefferson University Hospital (Philadelphia, Pa.) with Institutional Review Board approval. A total of 244 consecutive patients who underwent surgical resection from 1978 to 2008 and for whom tissue was available were included in this study. Clinical and treatment information were extracted via chart review. All patients were treated with surgical excision only (no radiation or hormonal therapy) by the same surgeon. Negative margins (≧10 mm) were achieved at the conclusion of excision or re-excision and removal of all suspicious calcifications was confirmed on postoperative mammography. Patients with DCIS involving more than 1 quadrant were treated by mastectomy and were excluded from this study. Patients who developed invasive ductal carcinoma within 6 months of a DCIS diagnosis were excluded because invasive ductal carcinoma was felt to be part of their original disease and not recurrence. The date of diagnosis and recurrence were defined as the date of surgery leading to the relevant pathologic diagnosis. The presence of DCIS, invasive cancer, or absence of disease at the last follow-up was established as the study endpoint. Median and mean follow up was 8.6 and 9.3 years respectively with 100 patients followed for over 10 years and 51 patients for over 15 years.

For each case, size, the histological pattern (cribriform, solid, comedo, papillary, micropapillary), the presence or absence of necrosis, and nuclear grade were evaluated. For determination of size of DCIS, the largest measurement of dimension from either the original report or review of histological sections was recorded. In some cases, size could not be assessed on review and was not recorded in the original histology report. Nuclear grade was assigned using established criteria¹⁸. Hormone receptor and HER2 status was obtained from pathology reports Immunohistochemical staining for estrogen receptor (clone SP1) progesterone receptor (clone 1E2), Ki67 (clone 30-9) and HER2 (clone 4B5) were performed on the BENCHMARK XT SLIDE PREPARATION SYSTEM™ (Ventana Medical Systems) using established clinical protocols and controls. Tissues were scored using ASCO/CAP guidelines¹⁹.

Immunohistochemistry:

Expression of PTEN and RB were assessed by employing a standard immunoperoxidase method with primary PTEN antibody (Cell Signaling Technologies, Rabbit Monoclonal, 138G6, 1:100) and primary RB antibody, (Thermoscientific; catalog no. MS-107-B, 1:50). Methods for RB and PTEN immunohistochemical staining have been described^{20,21}. RB expression was scored semi-quantitatively as negative (cancer cells showed no staining while normal cells were positive), weak (staining intensity was less than adjacent normal cells), or strong (staining intensity was equal to adjacent normal cells). RB-deficiency was defined as a score of either negative, or weak. PTEN expression was scored as negative or positive using published scoring criteria²¹. Evaluations were performed blinded to all clinical and biological variables. All stains were reviewed by two pathologists and disagreements were resolved by consensus.

Statistical Analysis:

Two time related endpoints were analyzed. First, endpoint was ipsilateral breast event (IBE) free survival, where failure is defined as the first reported DCIS recurrence or invasive breast carcinoma (IBC) recurrence. If the patient did not experience recurrence, she was classified as a censored observation on date of last follow-up. The Second endpoint was invasive breast carcinoma (IBC) recurrence free survival, where failure is defined as an invasive recurrence reported at any time. Follow up time was measured from the date of first DCIS surgery. The recurrence rates were estimated by Kaplan-Meier method and compared by log-rank test. The Cox proportional hazards models were utilized to determine the univariate and multivariable hazard ratios (HR) for standard clinical and pathological variables. A forward stepwise procedure with the criterion of p<0.05 was use to select individual variables for subsequent multivariate analysis. To test for interaction between PTEN and RB, a Cox model was used with the two main effects and the interaction term. P values less than 0.05 were considered statistically significant and were not adjusted for multiple testing. All analyses were performed with the use of SAS software (version 9.2; SAS Institute, Cary, N.C.). The statistical tests performed were two-sided.

Cell Culture and Immunoblotting:

Cells were maintained in DMEM/F12 supplemented with 5% Horse Serum, 100 μg/ml EGF, 10 ug/ml insulin, 100 ug/ml hydrocortisone, 100 U/ml penicillin/streptomycin and 2 mM L-glutamine. For challenge with low serum, cells were grown in media as described above with the exception of 0.5% Horse Serum without EGF. Cells were allowed to grow for three days before then processing for flow cytometry as described below. Additionally, cells were stained with 1% crystal violet to visualize outgrowth. For immunoblot analyses, equal total protein was separated by SDSPAGE. Proteins were detected by standard immunoblotting procedure using the following primary antibodies: Lamin B (M-20), pERK (E-4), ERK (K-32) (Santa Cruz Biotechnology, Santa Cruz, Calif., USA), PTEN (138G6) (Cell Signaling), and RB (G3-245) (Becton Dickson, Franklin Lakes, N.J., USA).

BrdU Labeling and Bivariate Flow Cytometry:

For cell proliferation analysis cells were incubated with bromodeoxyuridine (BrdU) (Amersham Pharmacia Biotech) for one hour before harvest. Cells were washed in PBS, and fixed in cold 70% ethanol. Bivariate flow cytometry was utilized for dual analysis of BrdU incorporation and total DNA content.

Three-Dimensional (3-D) Cultures of Mammary Epithelial Cells:

Cells were treated with trypsin and resuspended in assay medium (DMEM/F12 supplemented with 2% horse serum, 10 m/ml insulin, 100 m/ml hydrocortisone), 100 U/ml penicillin/streptomycin and 2 mM L-glutamine at a concentration of 25,000 cells per ml. Eight-chambered RS glass slides (Nalgene Nunc. Naperville, Ill.) were coated with 40 μl MATRIGEL™ (BD Bioscience Bedford, Mass., USA) and solidified for 30 min. The cells were mixed 1:1 with assay medium containing 4% MATRIGEL™ and 10 ng/ml EGF. 400 μl of the cell mixture was added to each well with 5,000 cells per chamber. Assay media was replaced every 4 days. Acini growth was monitored by imaging MCF10A miNS, MCF10A miRB, PTEN−/− miNS, and PTEN−/− miRB acini with a 20× objective. Diameters were measured at the middle optical section of each acinus, with the support of IMAGE J™ software.

Invasion Assay: MCF and PTEN−/− cells were seeded (5×10⁴ cells) Boyden Chambers (Franklin Lakes, N.J.; BioCoat 354578) under low serum conditions. Complete growth medium was added to the wells as the chemo-attractant. Chambers were placed in wells containing complete medium. The cells on the lower surface of the membrane were counterstained with 4,6-diamidino-2-phenylindole (DAPI) (Sigma-Aldrich, St. Louis, Mo., USA). Cells were scored with a fluorescent microscope.

### Results

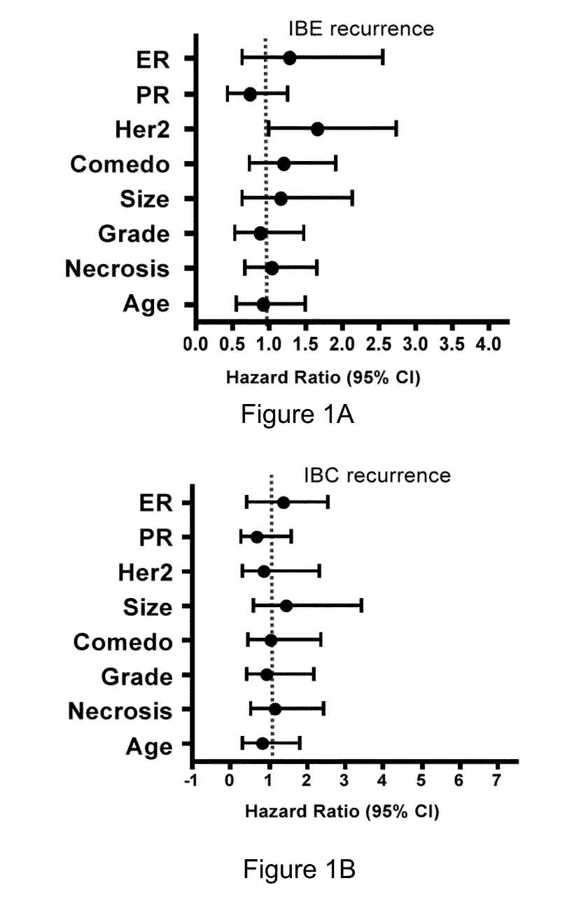

Prior studies have implicated the RB-pathway as a determinant of DCIS recurrence^{8,20,22}. These studies utilized p16ink4a and Ki67 as surrogates for loss of RB function^{8,20,22}. Surprisingly, no published studies have directly investigated the expression of the RB tumor suppressor protein in a cohort of DCIS cases with long-term follow up. Here, a cohort of 236 patients with DCIS were treated by surgical resection with wide-margins and subjected to long-term careful follow up (median 8.3 years) with tissue sufficient to perform staining for two markers; RB and PTEN. In this cohort the frequency of any ipsilateral breast recurrence was 32% (n=75) and the frequency of recurrent invasive disease was 11% (n=27). Multiple standard clinical and pathological variables were evaluated for recurrence (Table 1). As shown, age, necrosis, nuclear grade, comedo histology, ER-status, and PR-status were not significantly associated with IBE or IBC recurrence (Table 1 and FIGS. 1A-1B). High levels of HER2 were weakly associated with IBE recurrence (HR=1.66, p=0.048), but were not associated with IBC recurrence. These findings are largely consistent with other studies, and due to the absence of confounding effects of adjuvant therapy this cohort is an ideal one upon which to evaluate prognostic determinants of disease recurrence.

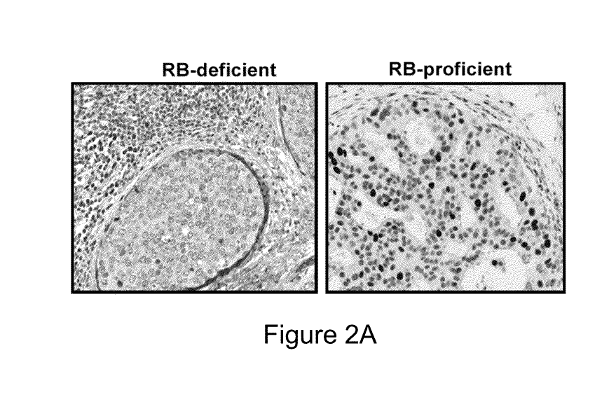

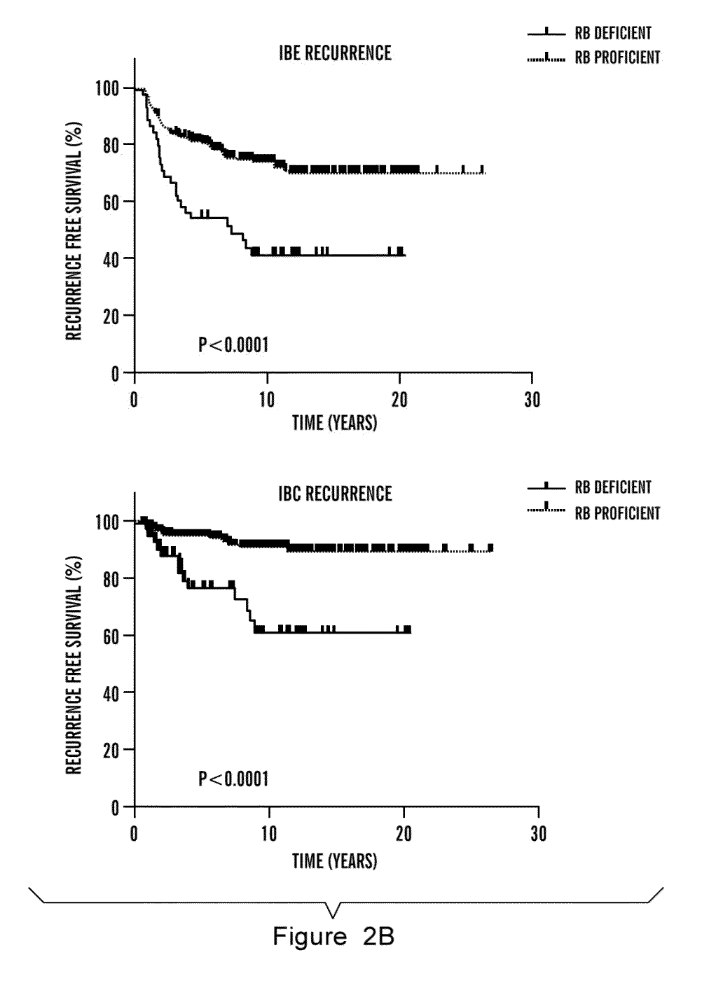

Immunohistochemical analyses of RB were optimized in a clinical laboratory and all staining was performed with positive and negative controls. For RB positive staining (RB proficient) the cells of the DCIS lesion retained levels of nuclear staining comparable to stroma or lymphocytes in the tissue (FIG. 2A). For staining defined as RB deficient there was negative or very weak nuclear staining within the tumor compartments, but surrounding cells were positive, serving as built in internal positive control. In the analyzed cohort, 19.5% percent of cases were RB deficient with 17.4% exhibiting negative and 1.7% very weak staining in reference to internal positive control. Kaplan-Meier analyses were performed on recurrence as a function of RB-status. These data revealed that compromised RB expression was significantly associated with IBE recurrence (HR=2.64 with p<0.0001, Table 2 and FIG. 2B). The estimated 15 year IBE recurrence rate in RB-deficient DCIS women was 58% as compared against 29% for RB proficient DCIS women. In the analyses of IBC there was an even stronger effect (FIG. 2C), with RB-proficient cases exhibiting a relatively low risk of undergoing invasive progression (8% at 10 years, 10% estimated at 15 years), while RB-deficient cases exhibited 39% recurrence as invasive lesions (HR 4.66 with p<0.0001, Table 2). These data remained significant in multivariate analyses against standard clinicopathological features of DCIS (Table 5).

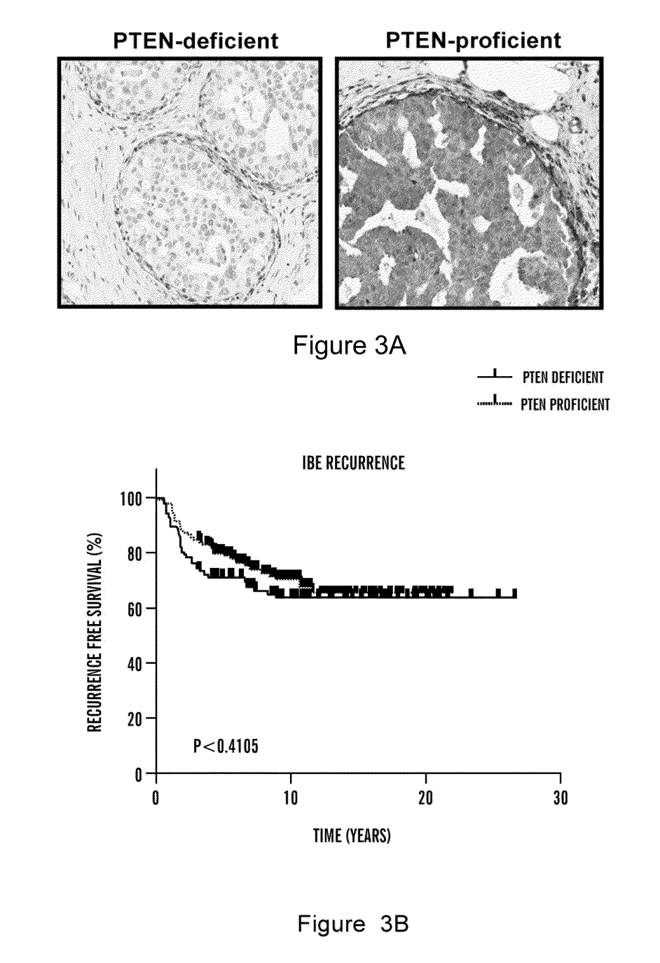

While the association of RB-status with recurrence and progression was significant, there were clearly RB-deficient tumors that failed to progress. In the analyses of RB negative breast cancer cases, it became clear that PTEN loss occurs at quite high-frequency, suggesting that cooperation between RB and PTEN loss may be particularly germane to aggressive disease (e.g. cases that are at increased risk to progress to invasive breast cancer). As with RB, the staining for PTEN was rigorously optimized with positive and negative controls (FIG. 3A). In the described cohort 44% of DCIS cases were PTEN deficient. In general, PTEN loss is believed to be associated with aggressive tumorigenic behavior. However, in the DCIS cohort analyzed PTEN-status was not associated with risk of IBE recurrence (p=0.41) or risk of IBC recurrence (p=0.31) (FIG. 3B and Table 3). Thus, PTEN loss is not a prognostic variable in DCIS.

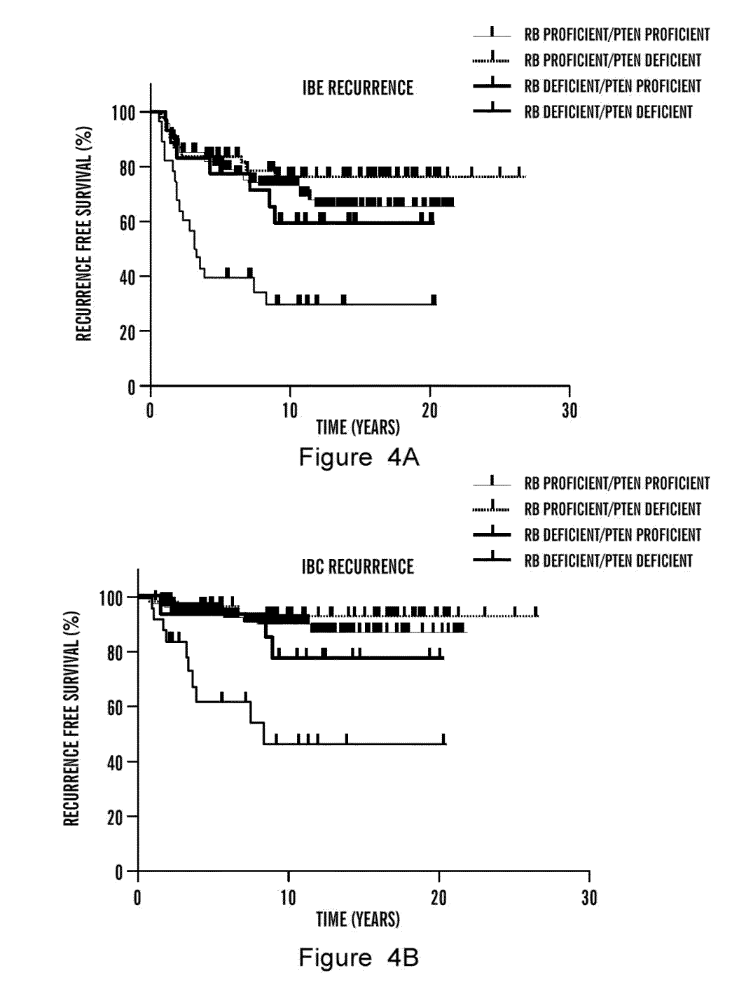

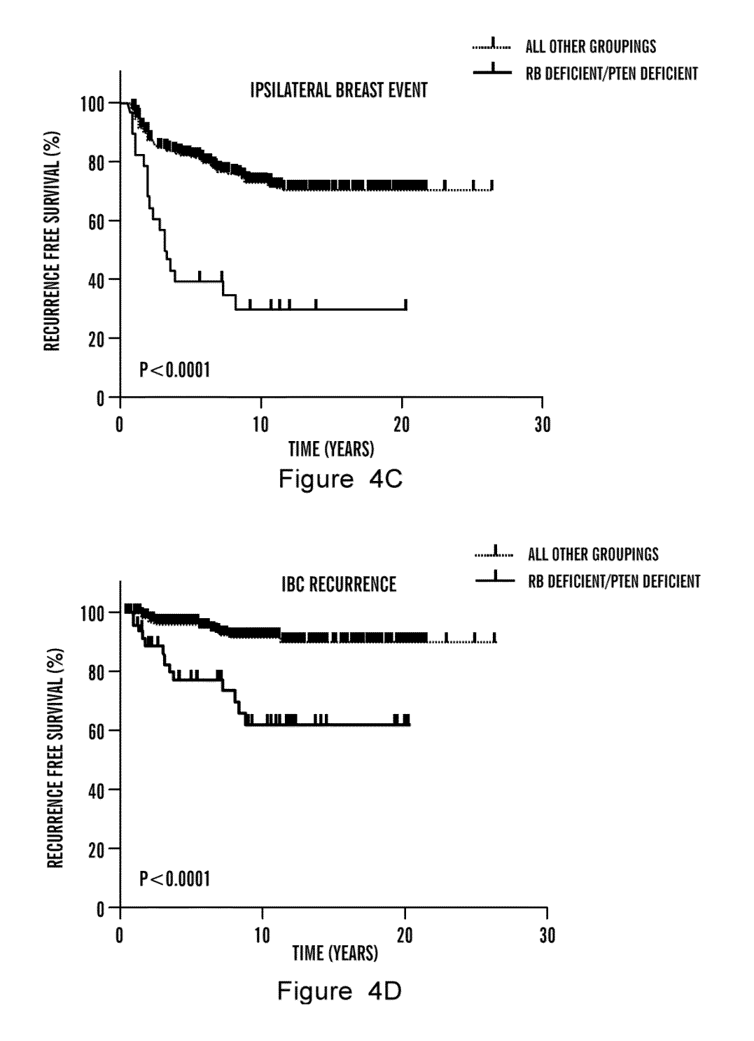

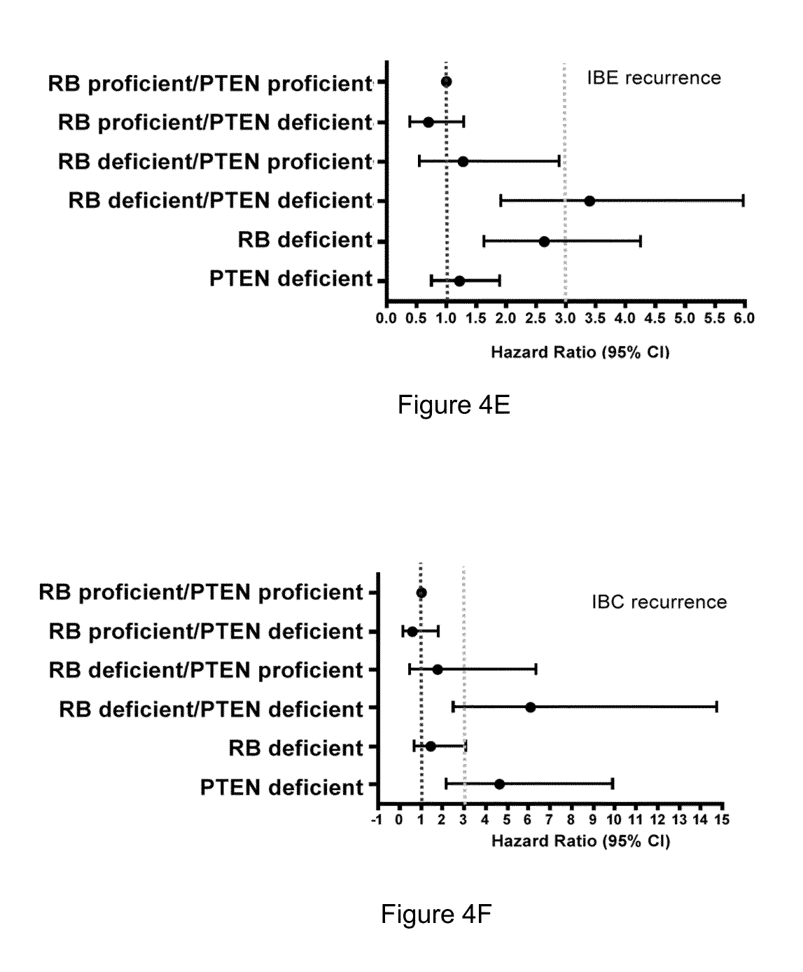

To specifically investigate the interaction between RB and PTEN, the cases were stratified into 4 groups based on combined RB/PTEN status. Kaplan-Meier recurrence free survival curves revealed that cases that were deficient for both RB and PTEN were at significantly increased risk of recurrence relative to all other groupings (FIG. 4A). As summarized in Table 3, in univariate analyses RB/PTEN compound loss was strongly associated with risk of IBE recurrence (HR 3.39), in contrast RB deficient yet PTEN positive DCIS was only modestly associated with increased risk of IBE recurrence (HR 1.28). Thus, PTEN status was highly relevant for defining RB-deficient tumors that would recur. Combined deficiency for RB and PTEN was even more strongly associated with risk of IBC recurrence (HR 6.10), and similarly discriminated from RB-deficient DCIS that were PTEN positive (HR 1.74). Statistical modeling demonstrated that there was a statistically significant interaction between RB and PTEN, and the combination of RB/PTEN as a determinant of recurrence was significant in multivariate analyses (Table 5). Women with the combined loss have extremely poor prognosis. At 2 and 5 years, their estimated rates of IBE recurrence was 36 and 61% and their rates of IBC recurrence of 16 and 38%. Together, these data indicate that the combination of RB and PTEN can be employed effectively to define DCIS at high risk of recurrence and progression to invasive disease (FIGS. 4E and 4F).

Of the 230 DOS cases, 68 (29%) recurred (46 as DCIS and 22 as invasive carcinoma). There was a statistically significant association between loss of RB expression and invasive recurrence (p=0.0014). However, its significance was increased when considered in combination with PTEN loss (p<0.0001). An invasive recurrence was observed in 41% of patients with RB PTEN loss, while 6% of patients with no recurrence showed RB/PTEN loss of expression.

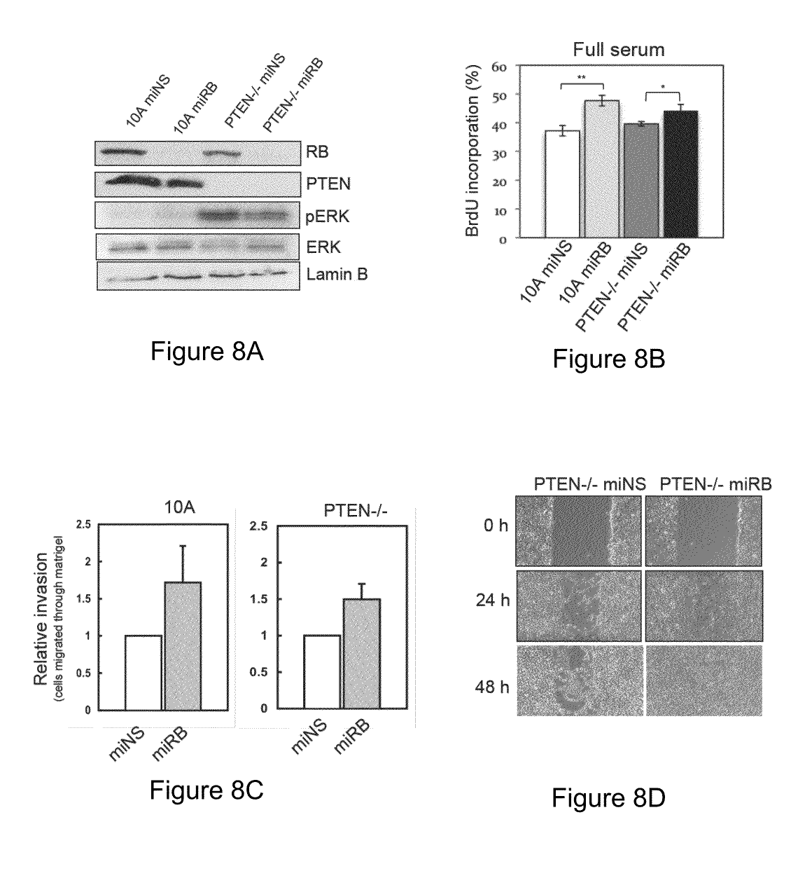

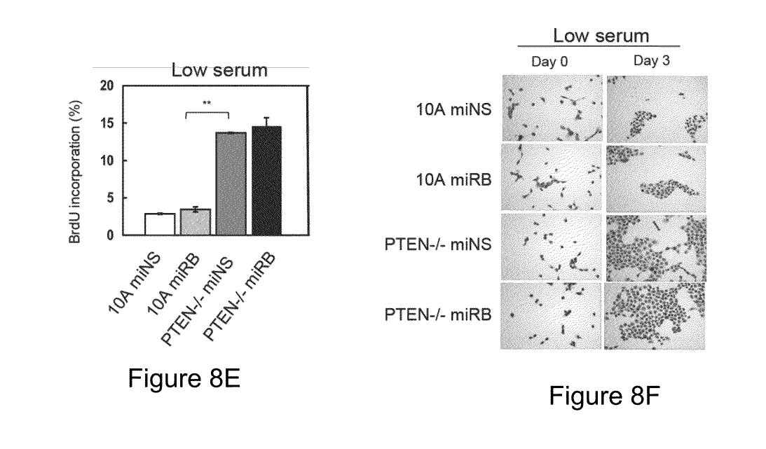

To determine the functional effect of RB and PTEN alone and in combination on DCIS progression, the loss of the tumor suppressors was modeled in MCF10A cells. The MCF10A model is an immortalized breast epithelia line that has been extensively utilized to dissect determinants of breast carcinogenesis. To model the inactivation of PTEN, matched cell lines with intact PTEN or PTEN deleted by homologous recombination were employed²⁴. In these cells, RB was depleted using established knockdown methodology (miRB)²⁰. As shown in FIG. 8A, effective deletion of PTEN and knockdown of RB was confirmed in these cell by immunoblotting. Furthermore, elevated ERK activity was observed with PTEN deletion consistent with published data²⁴. Thus, the four lines recapitulate the loss of RB and PTEN as is observed in DCIS. Because both RB and PTEN are implicated in cell cycle control, proliferation was evaluated by monitoring BrdU incorporation (FIG. 8B). In the context of MCF10A cells, PTEN-deficiency had no significant effect on BrdU incorporation in full serum (FIG. 8B). In contrast, RB loss led to a modest, yet statistically significant, increase in BrdU incorporation.

Since recurrence and progression likely reflect the ability of cells to disseminate away from the primary lesion, the motility and invasive properties of the cell populations were evaluated. Although PTEN has been implicated in invasion, it had little effect on the ability of cells to invade through matrigel in modified Boyden chamber assays (not shown). However, RB promoted a more invasive phenotype in both Boyden chamber and wound-healing assays (FIGS. 8C and 8D) which is consistent with prior studies^{20,25}. It is well known that RB-deficient cells retain a number of dependencies that limit the development of an invasive cancer. Interestingly, when the ability of the populations to grow under conditions of low growth factor (FIGS. 8E and 8F) or lack of adhesion (not shown) was tested, only PTEN loss provided an advantage to the cell populations. These data indicate differential and complementary effects of RB and PTEN loss on proliferative control in MCF10A cells.

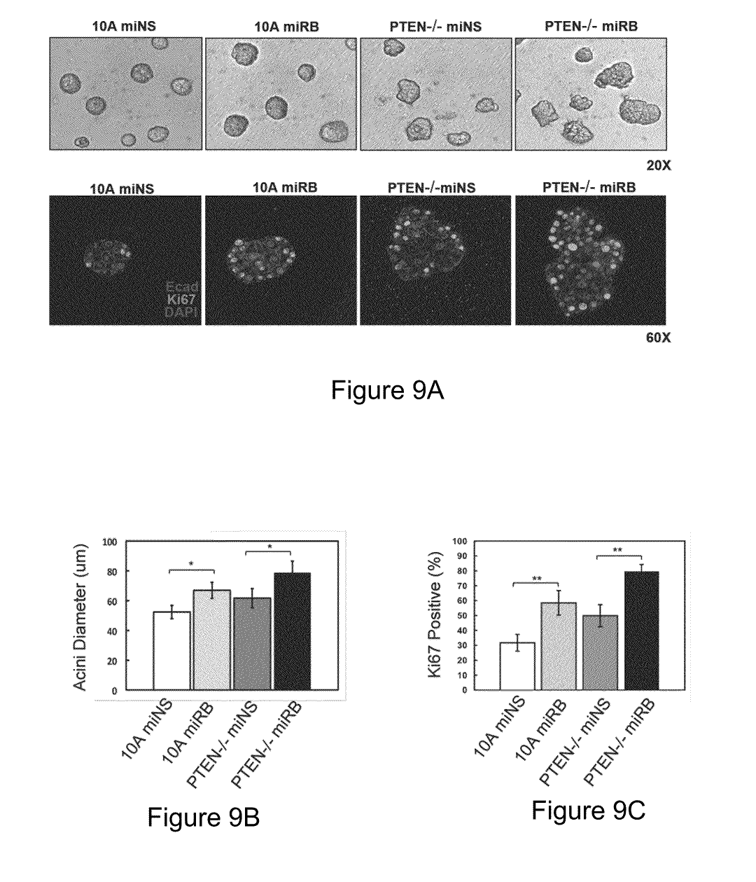

One means to evaluate multiple aspects of mammary cell biology is the analyses of 3D growth in matrigel^{26,27}. In this assay, RB loss leads to an acceleration of acinar growth and enhanced Ki67 staining (FIGS. 9A-9C). However, RB-deficient cells are still prone to cell death, and are cleared from the lumen resulting in formation of hollow acinar structures. As such, RB-deficient MCF10A populations retain a relatively normal overall morphology and organization (FIG. 9A). In contrast, PTEN loss facilities the survival of luminal cells and a degree of acinar disorganization not observed in RBdeficient cells (FIG. 9A). These effects were largely additive in reference to acinar size and proliferation (FIGS. 9B and 9C). Though individual loss of RB or PTEN have distinct effects on mammary epithelia, the combined loss results in a rapidly proliferating and invasive population. Without wishing to be bound by theory, these aspects likely contribute to the pronounced aggressiveness of RB and PTEN deficient DCIS and explain their prognostic significance in clinical samples.

### DISCUSSION

In the study described herein, two key tumor suppressors were evaluated for prognostic significance related to the recurrence and progression of DCIS. The findings described herein demonstrate that histological loss of RB protein expression is a strong independent marker of DCIS recurrence and progression to invasive cancer. While PTEN loss is frequently observed in DCIS, it had no significant association with disease outcome as a single variable. However, PTEN status served to effectively stratify RB-deficient cases that were either relatively indolent (PTEN-positive) or prone to recurrence/invasive progression (PTEN-negative). Together, these analyses demonstrate the importance of multi-marker analyses and permit a relatively simple means to detect high-risk DCIS.

The clinical management of DCIS changed significantly over last 3 decades. Initially DCIS lesions were treated by mastectomy¹. Subsequently, breast conserving surgery with radiation therapy and hormonal interventions became a standard treatment^{1,28,29}. However, it is unclear whether patients who have DCIS uniformly benefit from these interventions. In several studies, radiotherapy reduced in-situ or invasive recurrences by about 50%³⁰⁻³². Although radiotherapy is associated with substantial reductions in local recurrence, no differences have been reported in overall survival. Furthermore, since only 10-15% of cases recur as invasive disease without radiation therapy, clearly not all DCIS patients require radiation. Similarly, effects of adjuvant Tamoxifen are difficult to evaluate. In a recent analysis, adjuvant tamoxifen treatment conferred a decreased risk of contralateral, but not ispsilateral invasive breast cancer, which leaves the question of whether tamoxifen works through suppression of recurrence or second primary disease³³. Therefore, identifying DCIS cases that can be cured by surgery alone is critical for effectively managing disease and mitigating over-treatment of patients.

Currently, there are no clinically applied tests based on clinicopathologic features or biomarkers which allow for personalized treatment of DCIS^{22,34}. Due to the difficulty in obtaining sufficient material from banked DCIS tissue, gene expression profiling has significantly lagged behind that of invasive breast cancer. As such, most markers that have been associated with disease recurrence or invasive progression of DCIS have emerged from histological analyses of factors implicated in the pathogenesis of invasive breast cancer. For example, HER2 overexpression has been extensively investigated as a marker of recurrence and progression to invasive disease. The results from such studies remain in debate, and as shown here, HER2 overexpression alone is a relatively weak marker for invasive progression of DCIS.

Described herein is a highly optimized protocol for detecting loss of RB tumor suppressor protein expression in DCIS. As a single variable, loss of RB was significantly associated with overall recurrence and invasive progression in DCIS. Interestingly, while there is strong concordance between p16ink4a/Ki67 high expression and RB loss, there is not an absolute relationship. In the cohort described herein, RB loss was a stronger prognostic maker than the combination of p16ink4a and Ki67 high expression (not shown).

While the hazard ratios for RB loss were relatively strong, it was clear that a substantial fraction of women harboring RB-deficient tumors could be cured by surgery alone. This finding led to the investigation of other markers that could be used to further stratify RB-deficient cases. One of the pathways implicated in breast cancer etiology and progression is the PTEN/PI3KCA/AKT pathway. Additionally, germline PTEN mutations are found in the autosoma dominant Cowden syndrome, which is characterized by multiple hamartomas as well as an increased risk of breast cancer. PTEN loss can result from mutation, loss of heterozygosity (LOH), and epigenetic down-modulation, and has been reported in nearly 50% of human cancers including breast cancer³⁵. Studies evaluating PTEN expression by immunohistochemistry demonstrated loss or weak expression in 33% of invasive breasts cancer³⁶⁻³⁸. Furthermore, PTEN loss correlated with high tumor grade, larger tumor size, negative hormone receptor status and poor prognosis³⁶. Although PTEN influences prognosis of established malignancy, its role in the early stages of cancer development is less established. In one study investigating frequency of LOH in DCIS, LOH at the PTEN locus was only present in DCIS lesions coexisting with IBC and not in pure DCIS¹⁶. A recent study investigating PTEN promoter methylation status in DCIS demonstrated presence of PTEN hypermethylation in the subset of pure DCIS and DCIS associated with IBC. Surprisingly, as described herein, the histological loss of PTEN was relatively common in DCIS, it was not associated with recurrence or progression to invasive breast cancer. Therefore, although lost at relatively high-frequency, PTEN as a single marker has little prognostic significance in DCIS treated by surgery. Further, the data described herein demonstrate that markers and/or biomarker panels which may be predictive in IBC (i.e. PTEN alone) are not necessarily useful as biomarkers in subjects with DCIS.

For clinical utilization it is critical to develop panels of markers that have high specificity and sensitivity. By employing PTEN-status, the overall specificity of RB loss for IBE and IBC recurrence was significantly enhanced. Prior data regarding the cooperation versus redundancy of RB and PTEN is not clear as to the nature of the interaction between these two geneS^{17,39,40}. In the functional studies described herein, it was apparent that RB loss contributes to invasiveness and aberrant proliferation, while PTEN loss enhanced cell survival and the ability to proliferate under low exogenous growth factor concentration.

Given the specificity of combined RB and PTEN loss for invasive progression and recurrence it indicates that this “subtype” of DCIS would warrant more aggressive therapeutic intervention. Importantly, both RB and PTEN deficiency are associated with increased sensitivity to DNA damaging agents⁴³⁻⁴⁵. Thus, the findings described herein indicate that directing radiation therapy against the RB/PTEN deficient DCIS which harbors poor prognosis can be beneficial.

The use of loss of PTEN and RB expression as a coordinate marker for the risk of recurrence in subjects with DCIS is a significant improvement over known markers. For example, while loss of RB in a DCIS subject indicated a 58% chance that a subject would experience an ipsilateral breast event recurrence within 10 years (Table 2), the use of RB/PTEN coordinate loss permitted the identification of subjects having a 71% chance of experiencing an ispilateral breast event recurrence within 10 years (Table 4), an increase in predictive power of 23%. The risk of recurrence is also calculated herein using hazard ratios (e.g. a hazard ratio of 2 would indicate a 2-fold risk versus a control). For ipsilateral breast event recurrence, the hazard ratio for a subject with a RB-deficient tumor is 2.64 while a subject with an RB/PTEN-deficient tumor is calculated to have a hazard ratio of 3.39, a 28% increase in risk. For invasive recurrence, the hazard ratios are 4.66 (RB-deficient) and 6.1 (RB/PTEN-deficient), representing a 31% increase in risk. Clearly, the use of coordinate RB/PTEN loss is a significant improvement over existing markers. The increased predictive power can allow improved treatment and monitoring of high risk subjects while avoiding the undesired overtreatment of lower risk subjects. Further, the improved power of RB/PTEN coordinate loss as predictive when compared to known single markers is particularly surprising in light of the fact that PTEN is not in and of itself predictive in DCIS subjects.

### REFERENCES

1. Virnig B A, Tuttle T M, Shamliyan T, Kane R L. Ductal carcinoma in situ of the breast: a systematic review of incidence, treatment, and outcomes. J Natl Cancer Inst. Feb. 3 2010; 102(3):170-178.
2. Allegra C J, Aberle D R, Ganschow P, et al. National Institutes of Health State-of-the-Science Conference statement: Diagnosis and Management of Ductal Carcinoma In Situ Sep. 22-24, 2009. J Natl Cancer Inst. Feb. 3, 2010; 102(3):161-169.
3. Erbas B, Provenzano E, Armes J, Gertig D. The natural history of ductal carcinoma in situ of the breast: a review. Breast Cancer Res Treat. May 2006; 97(2):135-144.
4. Kuerer H M, Albarracin C T, Yang W T, et al. Ductal carcinoma in situ: state of the science and roadmap to advance the field. J Clin Oncol. Jan. 10 2009; 27(2):279-288.
5. Pinder S E, Duggan C, Ellis I O, et al. A new pathological system for grading DCIS with improved prediction of local recurrence: results from the UKCCCR/ANZ DCIS trial. Br J Cancer. Jun. 29 2010; 103(1):94-100.
6. Sanders M E, Schuyler P A, Dupont W D, Page D L. The natural history of low-grade ductal carcinoma in situ of the breast in women treated by biopsy only revealed over 30 years of long-term follow-up. Cancer. Jun. 15 2005; 103(12):2481-2484.
7. Hogue A, Sneige N, Sahin A A, et al. Her-2/neu gene amplification in ductal carcinoma in situ of the breast. Cancer Epidemiol Biomarkers Prev. June 2002; 11(6):587-590.
8. Gauthier M L, Berman H K, Miller C, et al. Abrogated response to cellular stress identifies DCIS associated with subsequent tumor events and defines basal-like breast tumors. Cancer Cell. November 2007; 12(5):479-491.
9. Cornfield D B, Palazzo J P, Schwartz G F, et al. The prognostic significance of multiple morphologic features and biologic markers in ductal carcinoma in situ of the breast: a study of a large cohort of patients treated with surgery alone. Cancer. Jun. 1 2004; 100(11):2317-2327.
10. Millar E K, Tran K, Man P, Graham P H. p27KIP-1, cyclin A and cyclin D1 protein expression in ductal carcinoma in situ of the breast: p27KIP-1 correlates with hormone receptor status but not with local recurrence. Pathol Int. April 2007; 57(4):183-189.
11. Nofech-Mozes S, Spayne J, Rakovitch E, et al. Biological Markers Predictive of Invasive Recurrence in DCIS. Clin Med Oncol. 2008; 2:7-18.
12. Oh Y L, Choi J S, Song S Y, et al. Expression of p21Waf1, p27Kip1 and cyclin D1 proteins in breast ductal carcinoma in situ: Relation with clinicopathologic characteristics and with p53 expression and estrogen receptor status. Pathol Int. February 2001; 51(2):94-99.
13. Witkiewicz A K, Rivadeneira D B, Ertel A, et al. Association of RB/p16-Pathway Perturbations with DCIS Recurrence Dependence on Tumor versus Tissue Microenvironment. Am J Pathol. Jul. 12. 2011.
14. Oliveira A M, Ross J S, Fletcher J A. Tumor suppressor genes in breast cancer: the gatekeepers and the caretakers. Am J Clin Pathol. December 2005; 124 Suppl:S 16-28.
15. Li J, Yen C, Liaw D, et al. PTEN, a putative protein tyrosine phosphatase gene mutated in human brain, breast, and prostate cancer. Science. Mar. 28 1997; 275(5308):1943-1947.
16. Bose S, Crane A, Hibshoosh H, Mansukhani M, Sandweis L, Parsons R. Reduced expression of PTEN correlates with breast cancer progression. Hum Pathol. April 2002; 33(4):405-409.
17. Chow L M, Endersby R, Zhu X, et al. Cooperativity within and among Pten, p53, and Rb pathways induces high-grade astrocytoma in adult brain. Cancer Cell. March 8; 19(3):305-316.
18. Lester S. Perspectives on margins in DCIS: pathology. J Natl Compr Canc Netw October; 8(10):1219-1222.
19. Hammond M E, Hayes D F, Dowsett M, et al. American Society of Clinical Oncology/College Of American Pathologists guideline recommendations for immunohistochemical testing of estrogen and progesterone receptors in breast cancer. J Clin Oncol. Jun. 1, 2010; 28(16):2784-2795.
20. Witkiewicz A K, Rivadeneira D B, Ertel A, et al. Association of RB/p16-pathway perturbations with DCIS recurrence: dependence on tumor versus tissue microenvironment. Am J Pathol. September, 2011; 179(3):1171-1178.
21. Sangale Z, Prass C, Carlson A, et al. A robust immunohistochemical assay for detecting PTEN expression in human tumors. Appl Immunohistochem Mol Morphol. March 2010; 19(2):173-183.
22. Kerlikowske K, Molinaro A M, Gauthier M L, et al. Biomarker expression and risk of subsequent tumors after initial ductal carcinoma in situ diagnosis. J Natl Cancer Inst. May 5, 2011; 102(9):627-637.
23. Rakovitch E, Nofech-Mozes S, Hanna W, et al. HER2/neu and Ki-67 expression predict non-invasive recurrence following breast-conserving therapy for ductal carcinoma in situ. Br J Cancer. Mar. 13, 2012; 106(6):1160-1165.
24. Vitolo M I, Weiss M B, Szmacinski M, et al. Deletion of PTEN promotes tumorigenic signaling, resistance to anoikis, and altered response to chemotherapeutic agents in human mammary epithelial cells. Cancer Res. Nov. 1 2009; 69(21):8275-8283.
25. Arima Y, Hayashi H, Sasaki M, et al. Induction of ZEB proteins by inactivation of RB protein is key determinant of mesenchymal phenotype of breast cancer. J Biol Chem. Mar. 9, 2012; 287(11):7896-7906.
26. Debnath J, Mills K R, Collins N L, Reginato M J, Muthuswamy S K, Brugge J S. The role of apoptosis in creating and maintaining luminal space within normal and oncogene expressing mammary acini. Cell. Oct. 4 2002; 111(1):29-40.
27. Debnath J, Muthuswamy S K, Brugge J S. Morphogenesis and oncogenesis of MCF-10A mammary epithelial acini grown in three-dimensional basement membrane cultures. Methods. July 2003; 30(3):256-268.
28. Hughes L L, Wang M, Page D L, et al. Local excision alone without irradiation for ductal carcinoma in situ of the breast: a trial of the Eastern Cooperative Oncology Group. J Clin Oncol. Nov. 10 2009; 27(32):5319-5324.
29. Fisher B, Dignam J, Wolmark N, et al. Lumpectomy and radiation therapy for the treatment of intraductal breast cancer: findings from National Surgical Adjuvant Breast and Bowel Project B-17. J Clin Oncol. February 1998; 16(2):441-452.
30. Wapnir I L, Dignam J J, Fisher B, et al. Long-term outcomes of invasive ipsilateral breast tumor recurrences after lumpectomy in NSABP B-17 and B-24 randomized clinical trials for DCIS. J Natl Cancer Inst. Mar. 16, 2010; 103(6):478-488.
31. Bijker N, Meijnen P, Peterse J L, et al. Breast-conserving treatment with or without radiotherapy in ductal carcinoma-in-situ: ten-year results of European Organisation for Research and Treatment of Cancer randomized phase III trial 10853—a study by the EORTC Breast Cancer Cooperative Group and EORTC Radiotherapy Group. J Clin Oncol. Jul. 20 2006; 24(21):3381-3387.
32. Houghton J, George W D, Cuzick J, Duggan C, Fentiman I S, Spittle M. Radiotherapy and tamoxifen in women with completely excised ductal carcinoma in situ of the breast in the UK, Australia, and New Zealand: randomised controlled trial. Lancet. Jul. 12 2003; 362(9378):95-102.
33. Cuzick J, Sestak I, Pinder S E, et al. Effect of tamoxifen and radiotherapy in women with locally excised ductal carcinoma in situ: long-term results from the UK/ANZ DCIS trial. Lancet Oncol. January 2010; 12(1):21-29.
34. Claus E B, Chu P, Howe C L, et al. Pathobiologic findings in DCIS of the breast: morphologic features, angiogenesis, HER-2/neu and hormone receptors. Exp Mol Pathol. June 2001; 70(3):303-316.
35. Vivanco I, Sawyers C L. The phosphatidylinositol 3-Kinase AKT pathway in human cancer. Nat Rev Cancer. July 2002; 2(7):489-501.
36. Depowski P L, Rosenthal S I, Ross J S. Loss of expression of the PTEN gene protein product is associated with poor outcome in breast cancer. Mod Pathol. July 2001; 14(7):672-676.
37. Perren A, Weng L P, Boag A H, et al. Immunohistochemical evidence of loss of PTEN expression in primary ductal adenocarcinomas of the breast. Am J Pathol. October 1999; 155(4):1253-1260.
38. FitzGerald MG, Marsh D J, Wahrer D, et al. Germline mutations in PTEN are an infrequent cause of genetic predisposition to breast cancer. Oncogene. Aug. 13 1998; 17(6):727-731.
39. Paramio J M, Navarro M, Segrelles C, Gomez-Casero E, Jorcano J L. PTEN tumour suppressor is linked to the cell cycle control through the retinoblastoma protein. Oncogene. Dec. 9 1999; 18(52):7462-7468.
40. Radu A, Neubauer V, Akagi T, Hanafusa H, Georgescu M M. PTEN induces cell cycle arrest by decreasing the level and nuclear localization of cyclin D1. Mol Cell Biol. September 2003; 23(17):6139-6149.
41. Jiang Z, Deng T, Jones R, et al. Rb deletion in mouse mammary progenitors induces luminal-B or basal-like/EMT tumor subtypes depending on p53 status. J Clin Invest. September 1; 120(9):3296-3309.
42. Knudsen E S, Ertel A, Davicioni E, Kline J, Schwartz G F, Witkiewicz A K. Progression of ductal carcinoma in situ to invasive breast cancer is associated with gene expression programs of EMT and myoepithelia. Breast Cancer Res Treat. Dec. 2, 2011.
43. Dedes K J, Wetterskog D, Mendes-Pereira A M, et al. PTEN deficiency in endometrioid endometrial adenocarcinomas predicts sensitivity to PARP inhibitors. Sci Transl Med. October 13; 2(53):53ra75.
44. Knudsen E S, Knudsen K E. Tailoring to RB: tumour suppressor status and therapeutic response. Nat Rev Cancer. Aug. 7 2008.
45. Knudsen E S, Wang J Y. Targeting the RB-pathway in cancer therapy. Clin Cancer Res. Feb. 15, 2010; 16(4):1094-1099.

TABLE 1

| | | | DCIS | Invasive | Hazard Ratio | Hazard Ratio |
|---|---|---|---|---|---|---|
| | Distribution | IBE | Recurrence | Recurrence | (IBE) | (IBC recurrence) |
| Variable | No. (%) | NO (%) | No. (%) | No. (%) | (95% CI) | (95% CI) |
| AGE | | | | | | |
| Under 50 | 69 (29%) | 23 (33%) | 14 (20%) | 9 (13%) | | |
| Over 50 | 167 (71%) | 52 (31%) | 34 (20%) | 18 (11%) | 0.91 (0.56, 1.49) | 0.82 (0.31, 1.82) |
| P-value | | | | | P = 0.72 | P = 0.63 |
| NECROSIS | | | | | | |
| No | 111 (47%) | 35 (32%) | 23 (21%) | 12 (11%) | | |
| Yes | 125 (53%) | 40 (32%) | 25 (20%) | 15 (12%) | 1.05 (0.67, 1.66) | 1.15 (0.54, 2.46) |
| P-value | | | | | P = 0.83 | P = 0.72 |
| NUCLEAR GRADE | | | | | | |
| 1 | 51 (22%) | 14 (28%) | 7 (14%) | 7 (14%) | | |
| 2 | 112 (47%) | 40 (36%) | 28 (25%) | 12 (11%) | | |
| 3 | 73 (31%) | 21 (29%) | 13 (18%) | 8 (11%) | 0.89 (0.54, 1.47) | 0.96 (0.42, 2.19) |
| P-value | | | | | P = 0.65 | P = 0.92 |
| TUMOR SIZE | | | | | | |
| Microfocal and | 113 (69%) | 34 (30%) | 20 (18%) | 14 (12%) | | |
| Focal and <1.0 cm | | | | | | |
| 1.00+ cm | 50 (31%) | 14 (32%) | 8 (16%) | 6 (16%) | 1.17 (0.65, 2.13) | 1.44 (0.60, 3.42) |
| P-value | | | | | P = 0.60 | P = 0.42 |
| COMEDO | | | | | | |
| No | 158 (67%) | 48 (30%) | 30 (19%) | 18 (11%) | | |
| Yes | 78 (33%) | 27 (35%) | 18 (23%) | 9 (12%) | 1.20 (0.75, 1.92) | 1.06 (0.48, 2.37) |
| P-value | | | | | P = 0.45 | P = 0.80 |
| HER2 | | | | | | |
| 0 | 65 (30%) | 19 (29%) | 13 (20%) | 6 (9%) | | |
| 1+ | 64 (30%) | 20 (32%) | 10 (16%) | 10 (16%) | | |
| 2+ | 25 (12%) | 4 (12%) | 2 (8%) | 2 (4%) | | |
| 3+ | 60 (28%) | 24 (4%) | 19 (32%) | 5 (8%) | 1.66 (1.01, 2.74) | 0.86 (0.32, 2.34) |
| P-value | | | | | P = 0.048 | P = 0.77 |
| PR | | | | | | |
| Negative | 49 (22%) | 18 (36%) | 11 (22%) | 7 (14%) | | |
| Positive | 174 (78%) | 54 (31%) | 35 (20%) | 19 (11%) | 0.74 (0.44, 1.27) | 0.67 (0.28, 1.60) |
| P-value | | | | | P = 0.27 | P = 0.37 |
| ER | | | | | | |
| Negative | 34 (15%) | 9 (27%) | 6 (18%) | 3 (9%) | | |
| Positive | 200 (85%) | 66 (33%) | 42 (21%) | 24 (12%) | 1.28 (0.64, 2.56) | 1.39 (0.42, 2.56) |
| P-value | | | | | P = 0.49 | P = 0.59 |

TABLE 2

| | RB-Deficient | RB-Proficient |
|---|---|---|
| IBE Recurrence Free Rate | | |
| Year 1 | 89% | 98% |
| Year 2 | 74% | 89% |
| Year 5 | 54% | 82% |
| Year 10 | 42% | 75% |
| Hazard Ratio | 2.64 (1.64, 4.25) | |
| P-Value | P < 0.0001 | |
| IBC Recurrence Free Rate | | |
| Year 1 | 96% | 99% |
| Year 2 | 88% | 97% |
| Year 5 | 77% | 96% |
| Year 10 | 61% | 92% |
| Hazard Ratio | 4.66 (2.19, 9.93) | |
| P-Value | P < 0.0001 | |

TABLE 3

| | PTEN-Deficient | PTEN-Proficient |
|---|---|---|
| IBE Recurrence Free Rate | | |
| Year 1 | 93% | 99% |
| Year 2 | 81% | 89% |
| Year 5 | 71% | 80% |
| Year 10 | 64% | 71% |
| Hazard Ratio | 1.21 (0.77, 1.90) | |
| P-Value | P = 0.41 | |
| IBC Recurrence Free Rate | | |
| Year 1 | 97% | 99% |
| Year 2 | 95% | 95% |
| Year 5 | 89% | 94% |
| Year 10 | 83% | 88% |
| Hazard Ratio | 1.47 (0.69, 3.13) | |
| P-Value | P = 0.31 | |

TABLE 4

| | RB-deficient/ | RB-deficient/ | RB-proficient/ | RB-proficient/ |
|---|---|---|---|---|
| | PTEN-deficient | PTEN-proficient | PTEN-deficient | PTEN-proficient |
| IBE Recurrence | | | | |
| Free Rate | | | | |
| Year 1 | 82% | 100% | 97% | 98% |
| Year 2 | 64% | 89% | 88% | 90% |
| Year 5 | 39% | 78% | 84% | 81% |
| Year 10 | 29% | 60% | 77% | 73% |
| Hazard Ratio | 3.39 (1.92, 5.99) | 1.28 (0.57, 2.90) | 0.71 (0.40, 1.31) | 1.00 |
| P-Value | P < 0.0001 | P = 0.55 | P = 0.28 | — |
| IBC Recurrence | | | | |
| Free Rate | | | | |
| Year 1 | 92% | 100% | 99% | 100% |
| Year 2 | 84% | 94% | 99% | 99% |
| Year 5 | 62% | 94% | 97% | 95% |
| Year 10 | 47% | 78% | 93% | 90% |
| Hazard Ratio | 6.1 (2.5, 14.76) | 1.74 (0.48, 6.36) | 0.58 (0.18, 1.84) | 1.00 |
| P-Value | P < 0.0001 | P = 0.40 | P = 0.35 | |

TABLE 5

| | | | |
|---|---|---|---|
| Variable | Subgroups | p-value | Hazard Ratio |

Forward selection with the Cox Model - IBE free survival

| RB | deficient vs proficient | <0.0001 | 2.64 (1.84, 4.25) |
|---|---|---|---|
| PTEN | deficient vs proficient | 0.63 | |
| HER2 (n = 214) | 0-2 vs 3 | 0.1 | |
| PR(n = 223) | Negative vs positive | 0.85 | |
| ER(n = 234) | Negative vs positive | 0.13 | |
| Age | <50 vs >50 years old | 0.87 | |
| Necrosis | No vs Yes | 0.89 | |
| Nuclear Grade | 1-2 vs 3 | 0.65 | |
| Comedo | No vs Yes | 0.85 | |
| Fixed Cox model with | RB and PTEN and interaction** | | |
| RB | deficient vs proficient | 0.55 | 1.28 (0.57, 2.90) |
| PTEN | deficient vs proficient | 0.28 | 0.72 (0.40, 1.31) |
| Interaction | RB-deficient/PTEN-deficient | 0.016 | 3.68 (1.28, 10.60) |
| | vs all others | | |

Forward selection with the Cox Model - IBC recurrence free survival

| RB | deficient vs proficient | P < 0.0001 | 4.66 (2.19, 9.93) |
|---|---|---|---|
| PTEN | deficient vs proficient | 0.47 | |
| HER2 (n = 214) | 0-2 vs 3 | 0.55 | |
| PR (n = 223) | Negative vs positive | 0.95 | |
| ER (n = 234) | Negative vs positive | 0.17 | |
| Age | <50 vs >50 years old | 0.95 | |
| Necrosis | No vs Yes | 0.97 | |
| Nuclear Grade | 1-2 vs 3 | 0.77 | |
| Comedo | No vs Yes | 0.66 | |
| Fixed Cox model with | RB and PTEN and interaction** | | |
| RB | deficient vs proficient | 0.72 | 1.75 (0.48, 6.36) |
| PTEN | deficient vs proficient | 0.82 | 0.58 (0.18, 1.83) |
| Interaction | RB-deficient/PTEN-deficient | 0.043 | 6.05 (1.06, 34.76) |
| | vs all others | | |

1. (canceled)

2. A method of administering a treatment for ductal carcinoma in situ (DCIS) to a subject, the method comprising:
subjecting a test sample of a subject to at least one analysis to determine the level of expression of both (a) retinoblastoma 1 (RB) and (b) phosphatase and tensin homolog (PTEN); and
administering a treatment for DCIS to the subject if the expression level of both RB and PTEN is decreased relative to a reference level;
wherein the treatment is not a lumpectomy.

3. The method of claim 2, wherein the treatment for ductal carcinoma in situ (DCIS) is selected from the group consisting of:
radiation; chemotherapy; tamoxifen; mastectomy; and radical mastectomy.

4. The method of claim 2, wherein the subject is identified as needing multiple treatments for DCIS if the expression level of both RB and PTEN is decreased relative to a reference level.

5. The method of claim 4, wherein the multiple treatments for ductal carcinoma in situ (DCIS) comprise:
(a) lumpectomy; and
(b) at least one further treatment selected from the group consisting of:
radiation; chemotherapy; tamoxifen; mastectomy; and radical mastectomy.

6. The method of claim 2, wherein the expression level of both (a) retinoblastoma 1 (RB) and (b) phosphatase and tensin homolog (PTEN) is the level of RNA transcript expression product of each gene.

7. The method of claim 6, wherein the RNA transcript expression product levels are assayed using reverse transcription polymerase chain reaction (RT-PCR).

8. The method of claim 2, wherein the expression level of both (a) retinoblastoma 1 (RB) and (b) phosphatase and tensin homolog (PTEN) is the level of polypeptide expression product of each gene.

9. The method of claim 8, wherein the polypeptide expression product levels are assayed using immunohistological staining.

10. The method of claim 8, wherein the level of polypeptide expression product of each gene is measured by an assay comprising:
(a) obtaining a test sample comprising a ductal carcinoma in situ (DCIS) cell from the breast tissue of a subject by lumpectomy or biopsy;
(b) contacting at least one portion of the sample with a primary anti-retinoblastoma 1 (RB) antibody and at least one portion of the sample with a primary anti-phosphatase and tensin homolog (PTEN) antibody;
(c) washing the sample to remove excess unbound primary antibody; and
(d) detecting the presence or intensity of a detectable signal;
wherein a decrease in the expression level of both RB and PTEN, indicated by the level of the detectable signal, relative to a reference level indicates the subject has a higher risk of experiencing an ipsilateral breast event.

11. The method of claim 10, wherein the primary antibody is detectably labeled or capable of generating a detectable signal.

12. The method of claim 10, wherein, between steps (c) and (d), the assay further comprises:
contacting the sample with a secondary antibody having a detectable label or capable of generating a detectable signal; and
washing the sample to remove excess unbound secondary antibody.

13. The method of claim 10, wherein one portion of the sample is contacted with both a primary anti-retinoblastoma 1 (RB) antibody and a primary anti-phosphatase and tensin homolog (PTEN) antibody.

14. The method of claim 10, wherein separate portions of the sample are contacted with a primary anti-retinoblastoma 1 (RB) antibody and a primary anti-phosphatase and tensin homolog (PTEN) antibody.

15. The method of claim 2, wherein the treatment is administered if the expression levels of both (a) retinoblastoma 1 (RB) and (b) phosphatase and tensin homolog (PTEN) are decreased by at least 50% in the test sample relative to the reference level.

16. The method of claim 2, wherein the expression level of both (a) retinoblastoma 1 (RB) and (b) phosphatase and tensin homolog (PTEN) are normalized relative to the expression level of one or more reference genes.

17. The method of claim 2, wherein the test sample comprises a ductal carcinoma in situ (DCIS) cell.

18. The method of claim 2, wherein the test sample is obtained by performing a lumpectomy or biopsy on the subject.

19. The method of claim 2, wherein the reference level of expression of (a) retinoblastoma 1 (RB) and (b) phosphatase and tensin homolog (PTEN) is the level of expression in non-cancerous tissue of the subject surrounding a DCIS cell.

20. The method of claim 2, wherein the reference level of expression of (a) retinoblastoma 1 (RB) and (b) phosphatase and tensin homolog (PTEN) is the level of expression in the breast tissue of a healthy subject.

21. The method of claim 2, wherein the expression level of no more than 20 other genes is determined.

22. The method of claim 2, wherein the expression level of no more than 10 other genes is determined.

23. The method of claim 2, wherein the subject is a human.
